SEQUENCE LISTING (23 sequences)

SEQ ID NO: 1 - protein, 489 aa, Rhodococcus sp.
MRRIPLSALIVAGSLILGGGIAAAQGSLPTSGSAGGTDVPPSYLTDDAGRSLILRGFNTASSAKSAPDGMPKFTEADLEREHADMGTNFVRFLISWRSVEPEPGQYDQEYLDRVEDRVSWYAERGYKVMLDMHQDVYSGAITPEGNSGNGAGNIGNGAPAWATYMDGLPVSPQDRWELYYIQPGVMRAFDNFWNTTGQHPELVEHYANAWQAVAERFAGNDTVVAYDLMNEPWGGSLQGPAFEGGPLAALYQRTTNAIREVDQDSWVCVAPQAVGVNQGVPSGLTKINDPRAGEQRIAYCPHLYPLPLDLGDGYSGLSRTLTDATIDTWRDSVEHIAGNVLGGVPVILAEFGLDTTLPGATDYIDRVYATARELGAGVSYWSSDPGPWGPYLPDGTQTLLVDTLNRPYPRAVAGTPTEWSSSVDRLQLTFTPNPAVTAPTEIYLPESGFPGGVDVAGADIVNWNPQSRLLTVETADDAGPVTVTVTPAS

SEQ ID NO: 2 - protein, 474 aa, synthetic construct
MGHHHHHHMQGSLPTSGSAGGTDVPPSYLTDDAGRSLILRGFNTASSAKSEPEPGQYDQEYLDRVEDRVSWYAERGYKVMLDMHQDVYSGAITPEGNSGNGAGNIGNGAPAWATYMDGLPVSPQDRWELYYIQPGVMRAFDNFWNTTGQHPELVEHYANAWQAVAERFAGNDTVVAYDLMNEPWGGSLQGPAFEGGPLAALYQRTTNAIREVDQDSWVCVAPQAVGVNQGVPSGLTKINDPRAGEQRIAYCPHLYPLPLDLGDGYSGLSRTLTDATIDTWRDSVEHIAGNVLGGVPVILAEFGLDTTLPGATDYIDRVYATARELGAGVSYWSSDPGPWGPYLPDGTQTLLVDTLNRPYPRAVAGTPTEWSSSVDRLQLTFTPNPAVTAPTEIYLPESGFPGGVDVAGADIVNWNPQSRLLTVEAPDGMPKFTEADLEREHADMGTNFVRFLISWRSVTADDAGPVTVTVTPAS

SEQ ID NO: 3 - protein, 489 aa, synthetic construct
MRRIPLSALIVAGSLILGGGIAAAQGSLPTSGSAGGTDVPPSYLTDDAGRSLILRGFNTASSAKSAPDGMPKFTEADLEREHADMGTNFVRFLISWRSVEPEPGQYDQEYLDRVEDRVSWYAERGYKVMLDMHQDVYSGAITPEGNSGNGAGNIGNGAPAWATYMDGLPVSPQDRWELYYIQPGVMRAFDNFWNTTGQHPELVEHYANAWQAVAERFAGNDTVVAYDLMNEPWGGSLQGPAFEGGPLAALYQRTTNAIREVDQDSWVCVAPQAVGVNQGVPSGLTKINDPRAGEQRIAYCPHLYPLPLDLGDGYSGLSRTLTDATIDTWRDSVEHIAGNVLGGVPVILASFGLDTTLPGATDYIDRVYATARELGAGVSYWSSDPGPWGPYLPDGTQTLLVDTLNRPYPRAVAGTPTEWSSSVDRLQLTFTPNPAVTAPTEIYLPESGFPGGVDVAGADIVNWNPQSRLLTVETADDAGPVTVTVTPAS

SEQ ID NO: 4 - protein, 474 aa, synthetic construct
MGHHHHHHMQGSLPTSGSAGGTDVPPSYLTDDAGRSLILRGFNTASSAKSEPEPGQYDQEYLDRVEDRVSWYAERGYKVMLDMHQDVYSGAITPEGNSGNGAGNIGNGAPAWATYMDGLPVSPQDRWELYYIQPGVMRAFDNFWNTTGQHPELVEHYANAWQAVAERFAGNDTVVAYDLMNEPWGGSLQGPAFEGGPLAALYQRTTNAIREVDQDSWVCVAPQAVGVNQGVPSGLTKINDPRAGEQRIAYCPHLYPLPLDLGDGYSGLSRTLTDATIDTWRDSVEHIAGNVLGGVPVILASFGLDTTLPGATDYIDRVYATARELGAGVSYWSSDPGPWGPYLPDGTQTLLVDTLNRPYPRAVAGTPTEWSSSVDRLQLTFTPNPAVTAPTEIYLPESGFPGGVDVAGADIVNWNPQSRLLTVEAPDGMPKFTEADLEREHADMGTNFVRFLISWRSVTADDAGPVTVTVTPAS

SEQ ID NO: 5 - protein, 482 aa, synthetic construct
MRRIPLSALIVAGSLILGGGIAAAQGSLPTSGSAGGTDVPPSYLTDDAGRSLILRGFNTASSAKSAPDGMPKFTEADLEREHADMGTNFVRFLISWRSVEPEPGQYDQEYLDRVEDRVSWYAERGYKVMLDMHQDVYSGAITPEGNIGNGAPAWATYMDGLPVSPQDRWELYYIQPGVMRAFDNFWNTTGQHPELVEHYANAWQAVAERFAGNDTVVAYDLMNEPWGGSLQGPAFEGGPLAALYQRTTNAIREVDQDSWVCVAPQAVGVNQGVPSGLTKINDPRAGEQRIAYCPHLYPLPLDLGDGYSGLSRTLTDATIDTWRDSVEHIAGNVLGGVPVILASFGLDTTLPGATDYIDRVYATARELGAGVSYWSSDPGPWGPYLPDGTQTLLVDTLNRPYPRAVAGTPTEWSSSVDRLQLTFTPNPAVTAPTEIYLPESGFPGGVDVAGADIVNWNPQSRLLTVETADDAGPVTVTVTPAS

SEQ ID NO: 6 - protein, 467 aa, synthetic construct
MGHHHHHHMQGSLPTSGSAGGTDVPPSYLTDDAGRSLILRGFNTASSAKSEPEPGQYDQEYLDRVEDRVSWYAERGYKVMLDMHQDVYSGAITPEGNIGNGAPAWATYMDGLPVSPQDRWELYYIQPGVMRAFDNFWNTTGQHPELVEHYANAWQAVAERFAGNDTVVAYDLMNEPWGGSLQGPAFEGGPLAALYQRTTNAIREVDQDSWVCVAPQAVGVNQGVPSGLTKINDPRAGEQRIAYCPHLYPLPLDLGDGYSGLSRTLTDATIDTWRDSVEHIAGNVLGGVPVILASFGLDTTLPGATDYIDRVYATARELGAGVSYWSSDPGPWGPYLPDGTQTLLVDTLNRPYPRAVAGTPTEWSSSVDRLQLTFTPNPAVTAPTEIYLPESGFPGGVDVAGADIVNWNPQSRLLTVEAPDGMPKFTEADLEREHADMGTNFVRFLISWRSVTADDAGPVTVTVTPAS

SEQ ID NO: 7 - protein, 474 aa, synthetic construct
MGHHHHHHMQGSLPTSGSAGGTDVPPSYLTDDAGRSLILRGFNTASSAKSEPEPGQYDQEYLDRVEDRVSWYAERGYKVMLDMHQDVYSGAITPEGNSGNGAGNIGNGAPAWATYMDGLPVSPQDRWELYYIQPGVMRAFDNFWNTTGQHPELVEHYANAWQAVAERFAGNDTVVAYDLMNEPWGGSLQGPAFEGGPLAALYQRTTNAIREVDQDSWVCVAPQAVGVNQGVPSGLTKINDPRAGEQRIAYCPHLYPLPLDLGYGYSGLSRTLTDATIDTWRDSVEHIAGNVLGGVPVILASFGLDTTLPGATDYIDRVYATARELGAGVSYWSSDPGPWGPYLPDGTQTLLVDTLNRPYPRAVAGTPTEWSSSVDRLQLTFTPNPAVTAPTEIYLPESGFPGGVDVAGADIVNWNPQSRLLTVEAPDGMPKFTEADLEREHADMGTNFVRFLISWRSVTADDAGPVTVTVTPAS

SEQ ID NO: 8 - protein, 467 aa, synthetic construct
MGHHHHHHMQGSLPTSGSAGGTDVPPSYLTDDAGRSLILRGFNTASSAKSEPEPGQYDQEYLDRVEDRVSWYAERGYKVMLDMHQDVYSGAITPEGNIGNGAPAWATYMDGLPVSPQDRWELYYIQPGVMRAFDNFWNTTGQHPELVEHYANAWQAVAERFAGNDTVVAYDLMNEPWGGSLQGPAFEGGPLAALYQRTTNAIREVDQDSWVCVAPQAVGVNQGVPSGLTKINDPRAGEQRIAYCPHLYPLPLDLGYGYSGLSRTLTDATIDTWRDSVEHIAGNVLGGVPVILASFGLDTTLPGATDYIDRVYATARELGAGVSYWSSDPGPWGPYLPDGTQTLLVDTLNRPYPRAVAGTPTEWSSSVDRLQLTFTPNPAVTAPTEIYLPESGFPGGVDVAGADIVNWNPQSRLLTVEAPDGMPKFTEADLEREHADMGTNFVRFLISWRSVTADDAGPVTVTVTPAS

SEQ ID NO: 9 - protein, 467 aa, synthetic construct
MGHHHHHHMQGSLPTSGSAGGTDVPPSYLTDDAGRSLILRGFNTASSAKSEPEPGQYDQEYLDRVEDRVSWYAERGYKVMLDMHQDVYSGAITPEGNIGNGAPAWATYMDGLPVSPQDRWELYYIQPGVMRAFDNFWNTTGQHPELVEHYANAWQAVAERFAGNDTVVAYDLMNEPWGGSLQGPAFEGGPLAALYQRTTNAIREVDQDSWVCVAPQAVGVNQGVPSGLTKINDPRAGEQRIAYCPHLYPLPLDLGFGYSGLSRTLTDATIDTWRDSVEHIAGNVLGGVPVILASFGLDTTLPGATDYIDRVYATARELGAGVSYWSSDPGPWGPYLPDGTQTLLVDTLNRPYPRAVAGTPTEWSSSVDRLQLTFTPNPAVTAPTEIYLPESGFPGGVDVAGADIVNWNPQSRLLTVEAPDGMPKFTEADLEREHADMGTNFVRFLISWRSVTADDAGPVTVTVTPAS

SEQ ID NO: 10 - protein, 467 aa, synthetic construct
MGHHHHHHMQGSLPTSGSAGGTDVPPSYLTDDAGRSLILRGFNTASSAKSEPEPGQYDQEYLDRVEDRVSWYAERGYKVMLDMHQDVYSGAITPEGNIGNGAPAWATYMDGLPVSPQDRWELYYIQPGVMRAFDNFWNTTGQHPELVEHYANAWQAVAERFAGNDTVVAYDLMNEPWGGSLQGPAFEGGPLAALYQRTTNAIREVDQDSWVCVAPQAVGVNQGVPSGLTKINDPRAGEQRIAYCPHLYPLPLDLGPGYSGLSRTLTDATIDTWRDSVEHIAGNVLGGVPVILASFGLDTTLPGATDYIDRVYATARELGAGVSYWSSDPGPWGPYLPDGTQTLLVDTLNRPYPRAVAGTPTEWSSSVDRLQLTFTPNPAVTAPTEIYLPESGFPGGVDVAGADIVNWNPQSRLLTVEAPDGMPKFTEADLEREHADMGTNFVRFLISWRSVTADDAGPVTVTVTPAS

SEQ ID NO: 11 - protein, 467 aa, synthetic construct
MGHHHHHHMQGSLPTSGSAGGTDVPPSYLTDDAGRSLILRGFNTASSAKSEPEPGQYDQEYLDRVEDRVSWYAERGYKVMLDMHQDVYSGAITPEGNIGNGAPAWATYMDGLPVSPQDRWELYYIQPGVMRAFDNFWNTTGQHPELVEHYANAWQAVAERFAGNDTVVAYDLMNEPWGGSLQGPAFEGGPLAALYQRTTNAIREVDQDSWVCVAPQAVGVNQGVPSGLTKINDPRAGEQRIAYCPHLYPLPLDLGQGYSGLSRTLTDATIDTWRDSVEHIAGNVLGGVPVILASFGLDTTLPGATDYIDRVYATARELGAGVSYWSSDPGPWGPYLPDGTQTLLVDTLNRPYPRAVAGTPTEWSSSVDRLQLTFTPNPAVTAPTEIYLPESGFPGGVDVAGADIVNWNPQSRLLTVEAPDGMPKFTEADLEREHADMGTNFVRFLISWRSVTADDAGPVTVTVTPAS

SEQ ID NO: 12 - protein, 467 aa, synthetic construct
MGHHHHHHMQGSLPTSGSAGGTDVPPSYLTDDAGRSLILRGFNTASSAKSEPEPGQYDQEYLDRVEDRVSWYAERGYKVMLDMHQDVYSGAITPEGNIGNGAPAWATYMDGLPVSPQDRWELYYIQPGVMRAFDNFWNTTGQHPELVEHYANAWQAVAERFAGNDTVVAYDLMNEPWGGSLQGPAFEGGPLAALYQRTTNAIREVDQDSWVCVAPQAVGVNQGVPSGLTKINDPRAGEQRIAYCPHLYPLPLDLGCGYSGLSRTLTDATIDTWRDSVEHIAGNVLGGVPVILASFGLDTTLPGATDYIDRVYATARELGAGVSYWSSDPGPWGPYLPDGTQTLLVDTLNRPYPRAVAGTPTEWSSSVDRLQLTFTPNPAVTAPTEIYLPESGFPGGVDVAGADIVNWNPQSRLLTVEAPDGMPKFTEADLEREHADMGTNFVRFLISWRSVTADDAGPVTVTVTPAS

SEQ ID NO: 13 - protein, 467 aa, synthetic construct; identical to SEQ ID NO: 10

SEQ ID NO: 14 - protein, 492 aa, synthetic construct
MRKTVVAFAAAIAACSAVLSSTTTSAAPPATPITTLQADGTHLVDGYGRTVLLHGVNNVDKDAPYLPAGETLTPQDIDILVRHGFNTVRLGTSFDALMPQRGQIDEAYLDRLTGVVDALTARGMHVLLDNHQDGLSKAWGGNGFPEWAIESRPREWEPNPGFPLYYLMPSLNAGWDEVWGNTHGALDHLGTALGALAERVEGKPGVMGIELLNEPWPGSRFLSCFPNGCPDFDRTYQAAMQKLTDAVRAQNPTIPVYWEPNVTWNQMMPSNLFAPPVTPALTTADVVFAPHDYCIPSQLAIYLGLPQALRGLCVPQQDLTWSNIDAITERANVPTVITEFGDGDPTVLKNTLARADERFIGWQYWHFGAGNATDPFLGEVGRQLVRTYPQATAGEPGRMIFDADNGDFAYRFTPRAATRPTEIFVSDLHYPDGYAVQVDGGQVTSAPGARIVTVVADGSGPVTVKINRPGSAGAEVPDGPIETSSSGSSGSS

SEQ ID NO: 15 - protein, 467 aa, synthetic construct
MAPPATPITTLQADGTHLVDGYGRTVLLHGVNNVDKDAPYLPAGETLTPQDIDILVRHGFNTVRLGTSFDALMPQRGQIDEAYLDRLTGVVDALTARGMHVLLDNHQDGLSKAWGGNGFPEWAIESRPREWEPNPGFPLYYLMPSLNAGWDEVWGNTHGALDHLGTALGALAERVEGKPGVMGIELLNEPWPGSRFLSCFPNGCPDFDRTYQAAMQKLTDAVRAQNPTIPVYWEPNVTWNQMMPSNLFAPPVTPALTTADVVFAPHDYCIPSQLAIYLGLPQALRGLCVPQQDLTWSNIDAITERANVPTVITSFGDGDPTVLKNTLARADERFIGWQYWHFGAGNATDPFLGEVGRQLVRTYPQATAGEPGRMIFDADNGDFAYRFTPRAATRPTEIFVSDLHYPDGYAVQVDGGQVTSAPGARIVTVVADGSGPVTVKINRPGSAGAEVPDGPIETSSSGSSGSS

SEQ ID NO: 16 - protein, 490 aa, synthetic construct
MRRTRLVSLIVTGSLVFGGGVAAAQSSLAASGSGSGSGTALTPSYLKDDDGRSLILRGFNTASSAKSAPDGMPQFTEADLAREYADMGTNFVRFLISWRSVEPAPGVYDQQYLDRVEDRVGWYAERGYKVMLDMHQDVYSGAITPEGNSGNGAGAIGNGAPAWATYMDGLPVEPQPRWELYYIQPGVMRAFDNFWNTTGKHPELVEHYAKAWRAVADRFADNDAVVAYDLMNEPFGGSLQGPAFEAGPLAAMYQRTTDAIRQVDQDTWVCVAPQAIGVNQGLPSGLTKIDDPRAGQQRIAYCPHLYPLPLDIGDGHEGLARTLTDVTIDAWRANTAHTARVLGDVPIILGEFGLDTTLPGARDYIERVYGTAREMGAGVSYWSSDPGPWGPYLPDGTQTLLVDTLNKPYPRAVAGTPTEWSSTSDRLQLTIEPDAAITAPTEIYLPEAGFPGDVHVEGADVVGWDRQSRLLTVRTPADSGNVTVTVTPAA

SEQ ID NO: 17 - protein, 481 aa, synthetic construct
MGSSHHHHHHSSGLVPRGSHMSGSGSGSGTALTPSYLKDDDGRSLILRGFNTASSAKSAPDGMPQFTEADLAREYADMGTNFVRFLISWRSVEPAPGVYDQQYLDRVEDRVGWYAERGYKVMLDMHQDVYSGAITPEGNSGNGTGAIGNGAPAWATYMDGLPVEPQPRWELYYIQPGVMRAFDNFWNTTGKHPELVEHYAKAWRAVADRFADNDAVVAYDLMNEPFGGSLQGPAFEAGPLAAMYQRTTDAIRQVDQDTWVCVAPQAIGVNQGLPSGLTKIDDPRAGQQRIAYCPHLYPLPLDIGYGHEGLARTLTDVTIDAWRANTAHTARVLGDVPIILGSFGLDTTLPGARDYIERVYGTAREMGAGVSYWSSDPGPWGPYLPDGTQTLLVDTLNKPYPRAVAGTPTEWSSTSDRLQLTIEPDAAITAPTEIYLPEAGFPGDVHVEGADVVGWDRQSRLLTVRTPADSGNVTVTVTPAA

SEQ ID NO: 18 - protein, 474 aa, synthetic construct
MGSSHHHHHHSSGLVPRGSHMSGSGSGSGTALTPSYLKDDDGRSLILRGFNTASSAKSAPDGMPQFTEADLAREYADMGTNFVRFLISWRSVEPAPGVYDQQYLDRVEDRVGWYAERGYKVMLDMHQDVYSGAITPEGAIGNGAPAWATYMDGLPVEPQPRWELYYIQPGVMRAFDNFWNTTGKHPELVEHYAKAWRAVADRFADNDAVVAYDLMNEPFGGSLQGPAFEAGPLAAMYQRTTDAIRQVDQDTWVCVAPQAIGVNQGLPSGLTKIDDPRAGQQRIAYCPHLYPLPLDIGYGHEGLARTLTDVTIDAWRANTAHTARVLGDVPIILGSFGLDTTLPGARDYIERVYGTAREMGAGVSYWSSDPGPWGPYLPDGTQTLLVDTLNKPYPRAVAGTPTEWSSTSDRLQLTIEPDAAITAPTEIYLPEAGFPGDVHVEGADVVGWDRQSRLLTVRTPADSGNVTVTVTPAA

SEQ ID NO: 19 - DNA, 1470 bp, synthetic construct
  source 1..1470: mol_type = other DNA
gtgcgccgtatcccgctctcagcattgatagtcgcgggctcactgatactgggcggcggcattgccgcggctcagggcagcctgcccacgtcggggagcgccggaggcacggacgtgccgccgtcgtacctgaccgacgacgccggccgctccttgatcctgcgagggttcaacacggcgtcgagcgcaaaaagcgcacccgacggcatgccgaagttcaccgaagccgacctcgagcgcgaacatgcagacatgggaacaaatttcgtgcggttcctgatctcctggcgttcggtcgaaccggaaccaggacagtacgaccaggagtatctcgaccgcgtcgaggaccgggtcagctggtatgccgagcgcggctacaaggtcatgctcgacatgcatcaagacgtctattccggcgcgatcacaccagagggcaacagcggcaacggcgccggcaacatcggcaacggtgccccggcctgggcgacatacatggacggccttccggtcagccctcaagaccgctgggagctgtactacatccaacccggcgtgatgcgcgcattcgacaacttctggaacaccaccggccaacaccccgaactggtcgagcactacgcgaacgcatggcaggcggtggccgaacgttttgcaggcaatgacaccgtcgtggcatacgacctgatgaacgagccgtggggcggttccctccaaggccccgcattcgaaggcgggcccctcgctgccctgtatcagcgaaccaccaacgccatcagggaagtcgatcaggacagctgggtgtgcgtggccccgcaggccgtcggtgtcaaccaaggtgtccccagcggactcacgaagatcaacgacccgcgagccggcgaacagcgaatcgcctactgcccacacctgtacccactgcccctcgacctcggtgacggatactccggcctctcacgcacactcaccgacgcgaccatcgacacctggcgcgacagcgtcgaacacatcgccggaaacgtactcggcggcgtcccggtcattctcgccgaattcgggctcgacacaacactgcccggcgcaaccgactacatcgaccgcgtctacgcgaccgcccgagaactcggcgcgggcgtctcgtactggtccagtgatccggggccttggggcccgtacctgccggacggaacacagacactcctcgtcgacaccctgaacaggccgtatccgcgtgccgtcgctggcacacccaccgaatggtcctcgagcgttgaccgccttcagttgacgttcaccccgaaccccgcggtgacggcaccgaccgagatctacctacccgagagcggctttcccggcggcgtcgacgtcgcaggcgccgacatcgtgaactggaacccccaaagccggcttctcacagtggaaaccgcggacgacgcgggccccgtcacagtcacggtgacgccggcaagctga

SEQ ID NO: 20 - DNA, 1401 bp, synthetic construct
  source 1..1401: mol_type = other DNA
atgcaagggtcccttccaacctcagggtctgcaggcggaaccgacgtgccgccctcttaccttacggatgatgctggtcgcagtctgatccttcgcggttttaatacagcctcctctgcaaaaagcgctcctgacggcatgcccaagttcactgaggctgatttggaacgcgagcatgcagatatgggcaccaattttgtacgctttttgatctcgtggcgctctgtagaacccgaaccaggacagtacgaccaggaatacctggaccgcgttgaggaccgcgtttcatggtacgccgagcgcggctacaaagtaatgttggacatgcaccaagacgtgtactctggggctatcacacccgagggaaattccggtaacggagcggggaacatcggtaacggggcgcctgcttgggcaacatatatggacgggttgcccgtctcgcctcaggaccgctgggagctttactatatccaacccggggtcatgcgcgcttttgataacttttggaacaccacagggcagcaccccgagttagttgagcattacgccaatgcatggcaagctgttgctgaacgcttcgcggggaatgacacagttgtagcgtacgatctgatgaacgaaccgtggggcggatcgttacagggtccggcattcgaagggggcccacttgccgctctgtaccaacgtacgaccaacgcgatccgcgaagtcgatcaagattcatgggtatgcgttgcgccacaagcagtaggcgtgaatcagggagtcccctctggtttgacgaaaatcaatgacccacgcgctggggagcagcgcatcgcttattgtccacacctttaccctctgccgcttgatctgggtgacgggtactcagggctttcgcgtacgcttacggatgctacaatcgacacttggcgtgattctgtagagcacattgctggaaacgtgcttggaggagtaccggtcatcctggcaagctttggtctggatactacgttaccgggcgccacggactacattgaccgtgtctatgctaccgctcgcgagttgggtgcgggagtttcttattggtctagcgaccctgggccatggggtccatatcttcctgacgggacgcagaccttattagtggacaccttgaatcgcccttatccccgcgcagtagccgggacgccaacggagtggtctagctccgtcgatcgtctgcaactgacattcaccccaaaccctgcggttactgcacccacggaaatttaccttcccgaatcaggattcccgggcggcgtggacgttgcgggggctgacattgtgaactggaaccctcagtctcgtcttcttaccgtagaaactgcggacgatgctggtccagtcacggttactgtaacaccagcaagctga

SEQ ID NO: 21 - DNA, 1380 bp, synthetic construct
  source 1..1380: mol_type = other DNA
atgcaagggtcccttccaacctcagggtctgcaggcggaaccgacgtgccgccctcttaccttacggatgatgctggtcgcagtctgatccttcgcggttttaatacagcctcctctgcaaaaagcgctcctgacggcatgcccaagttcactgaggctgatttggaacgcgagcatgcagatatgggcaccaattttgtacgctttttgatctcgtggcgctctgtagaacccgaaccaggacagtacgaccaggaatacctggaccgcgttgaggaccgcgtttcatggtacgccgagcgcggctacaaagtaatgttggacatgcaccaagacgtgtactctggggctatcacacccgagggaaacatcggtaacggggcgcctgcttgggcaacatatatggacgggttgcccgtctcgcctcaggaccgctgggagctttactatatccaacccggggtcatgcgcgcttttgataacttttggaacaccacagggcagcaccccgagttagttgagcattacgccaatgcatggcaagctgttgctgaacgcttcgcggggaatgacacagttgtagcgtacgatctgatgaacgaaccgtggggcggatcgttacagggtccggcattcgaagggggcccacttgccgctctgtaccaacgtacgaccaacgcgatccgcgaagtcgatcaagattcatgggtatgcgttgcgccacaagcagtaggcgtgaatcagggagtcccctctggtttgacgaaaatcaatgacccacgcgctggggagcagcgcatcgcttattgtccacacctttaccctctgccgcttgatctgggtgacgggtactcagggctttcgcgtacgcttacggatgctacaatcgacacttggcgtgattctgtagagcacattgctggaaacgtgcttggaggagtaccggtcatcctggcaagctttggtctggatactacgttaccgggcgccacggactacattgaccgtgtctatgctaccgctcgcgagttgggtgcgggagtttcttattggtctagcgaccctgggccatggggtccatatcttcctgacgggacgcagaccttattagtggacaccttgaatcgcccttatccccgcgcagtagccgggacgccaacggagtggtctagctccgtcgatcgtctgcaactgacattcaccccaaaccctgcggttactgcacccacggaaatttaccttcccgaatcaggattcccgggcggcgtggacgttgcgggggctgacattgtgaactggaaccctcagtctcgtcttcttaccgtagaaactgcggacgatgctggtccagtcacggttactgtaacaccagcaagctga

SEQ ID NO: 22 - DNA, 1380 bp, synthetic construct
  source 1..1380: mol_type = other DNA
atgcaagggtcccttccaacctcagggtctgcaggcggaaccgacgtgccgccctcttaccttacggatgatgctggtcgcagtctgatccttcgcggttttaatacagcctcctctgcaaaaagcgctcctgacggcatgcccaagttcactgaggctgatttggaacgcgagcatgcagatatgggcaccaattttgtacgctttttgatctcgtggcgctctgtagaacccgaaccaggacagtacgaccaggaatacctggaccgcgttgaggaccgcgtttcatggtacgccgagcgcggctacaaagtaatgttggacatgcaccaagacgtgtactctggggctatcacacccgagggaaacatcggtaacggggcgcctgcttgggcaacatatatggacgggttgcccgtctcgcctcaggaccgctgggagctttactatatccaacccggggtcatgcgcgcttttgataacttttggaacaccacagggcagcaccccgagttagttgagcattacgccaatgcatggcaagctgttgctgaacgcttcgcggggaatgacacagttgtagcgtacgatctgatgaacgaaccgtggggcggatcgttacagggtccggcattcgaagggggcccacttgccgctctgtaccaacgtacgaccaacgcgatccgcgaagtcgatcaagattcatgggtatgcgttgcgccacaagcagtaggcgtgaatcagggagtcccctctggtttgacgaaaatcaatgacccacgcgctggggagcagcgcatcgcttattgtccacacctttaccctctgccgcttgatctgggttatgggtactcagggctttcgcgtacgcttacggatgctacaatcgacacttggcgtgattctgtagagcacattgctggaaacgtgcttggaggagtaccggtcatcctggcaagctttggtctggatactacgttaccgggcgccacggactacattgaccgtgtctatgctaccgctcgcgagttgggtgcgggagtttcttattggtctagcgaccctgggccatggggtccatatcttcctgacgggacgcagaccttattagtggacaccttgaatcgcccttatccccgcgcagtagccgggacgccaacggagtggtctagctccgtcgatcgtctgcaactgacattcaccccaaaccctgcggttactgcacccacggaaatttaccttcccgaatcaggattcccgggcggcgtggacgttgcgggggctgacattgtgaactggaaccctcagtctcgtcttcttaccgtagaaactgcggacgatgctggtccagtcacggttactgtaacaccagcaagctga

SEQ ID NO: 23 - DNA, 1446 bp, synthetic construct
  source 1..1446: mol_type = other DNA
atggggagcagccaccaccatcaccaccacagtagcggcctggttccgcgcggaagccacatgtcaggcagcgggagcggctcgggaacggccttaacgccgagctatttaaaagatgatgacggacgctcgctcattttacgtgggttcaacaccgcctcgtctgctaaaagtgcaccagacggaatgccgcaatttactgaagctgatttggcgcgtgaatatgcagatatggggaccaatttcgtacgctttctcatcagctggcgtagcgtggaaccagcaccaggtgtatatgatcagcagtatctggaccgcgtagaagatcgtgtaggatggtacgcagagcgtggttataaagtaatgctggatatgcatcaggacgtgtattctggcgcgattaccccggaagggaacagtggcaatggcacgggcgcgattgggaatggtgcaccggcatgggccacctatatggacggtctgccagtggagccgcagccgcgctgggaactgtattatattcaaccgggggtaatgcgtgcatttgacaatttttggaatacgacgggtaagcacccggaactcgtggaacactacgcaaaagcctggcgtgccgtggccgatcgtttcgcagacaatgatgctgtggttgcctatgacctgatgaacgaaccatttgggggctccctgcagggtccagcgttcgaagcagggccgctggcggcaatgtatcagcgcacaacggatgccatccgccaggtggaccaggatacgtgggtttgcgtcgccccgcaggcgattggcgtgaatcaagggctgccgagcggcctgaccaaaatcgacgatccgcgtgccggccaacaacgtattgcctactgcccgcatctgtacccgttaccgttagacattgggtatggccatgagggtctggcacgcaccttgaccgacgtaaccatcgacgcgtggcgcgcgaatactgcccacacggcacgcgttctgggtgacgtgccgattatcctgggctcattcggactcgacaccactctcccgggagcccgcgattatatcgaacgtgtgtatgggaccgcacgcgagatgggtgctggggtctcctattggagcagcgatccgggcccatgggggccgtatctgccggatggcacgcaaacccttttagttgacacactgaataaaccgtatccgcgtgccgtcgccggcactccgaccgagtggtcgtcgacctctgaccgtctgcagcttaccattgaaccggacgctgccattaccgccccgactgagatctatctgccggaggcgggctttccgggggatgtccatgttgaaggagcggacgttgtgggttgggaccgtcagtctcgtctgctcaccgtgcgtacgccggctgactcaggcaatgttaccgtaaccgtaaccccagccgcctaa

(19) United States
(12) Patent Application Publication
(10) Pub. No.: US 2025/0354131 A1
(43) Pub. Date: Nov. 20, 2025

(54) NOVEL GLYCOSYNTHASE

(71) Applicant: CarboCode S.A., Coimbra (PT)

(72) Inventors: Iria GRUNDLING, Coimbra (PT); Alexander DENNIG, Coimbra (PT); Matthias NACHSCHATT, Coimbra (PT); Halina ZHYLITSKAYA, Coimbra (PT); Györgyi OSZTROVSZKY, Coimbra (PT); Ferenc HORVATH, Coimbra (PT)

(21) Appl. No.: 18/724,574

(22) PCT Filed: Dec. 21, 2022
(86) PCT No.: PCT/EP2022/087366

§ 371 Date: Jun. 26, 2024

(30) Foreign Application Priority Data
Dec. 21, 2021 (PT) 117660

(51) Int. Cl. C12N 9/24; C12P 7/6436

(52) U.S. Cl. CPC C12N 9/2402; C12P 7/6436; C12Y 302/01123

(57) ABSTRACT

The present invention relates to a novel glycosynthase, especially an endoglycoceramide synthase, and to a method for the glycosylation of sphingolipids.

### CROSS-REFERENCE TO RELATED APPLICATIONS

This application is a US national stage entry of PCT/EP2022/087366 filed on 21 Dec. 2022, which claims priority from Portugal Application No. 117660 filed on 21 Dec. 2021, the contents of which are to be taken as incorporated herein by this reference.

The present invention relates to a novel glycosynthase, especially an endoglycoceramide synthase, for the glycosylation of sphingolipids. The invention further relates to a method for producing glycosphingolipids and to a nucleic acid encoding a glycosynthase, especially an endoglycoceramide synthase.

### REFERENCE TO SEQUENCE LISTING

The computer-readable Sequence Listing submitted on Feb. 17, 2025, and identified as follows: 36,392 bytes ST.26 XML file named “032603-808 Sequence_Listing.xml” created Feb. 17, 2025, is incorporated herein by reference in its entirety.

### BACKGROUND

Glycosylation reactions are widespread in nature and are involved in almost all vital processes. Glycoconjugates directly exert a wide range of functions, including energy storage, maintenance of cell structural integrity, information storage and transfer, molecular recognition, cell-cell interaction, cellular regulation, immune response, virulence and chemical defense. Glycoconjugates are the structurally most diverse biomolecules and their biosynthesis needs quite complex biological processes orchestrated by many enzyme systems.

Glycosphingolipids (GSLs) are a class of glycolipids mainly found on the surface of eukaryotic cells. Their structure consists of a glycan moiety conjugated to a sphingolipid unit. Owing to the diversity of the glycan moiety, GSLs represent a large family of glycoconjugates and to date more than 300 different structures have been identified.

GSLs are involved in diverse biological processes and play important structural and functional roles. For instance, they contribute to cell-cell recognition, communication, and intercellular adhesion. They have been shown to be involved in diverse immune processes as well as cancer angiogenesis and progression. Furthermore, certain GSLs are found in the brain and play roles in neurological diseases.

Because of their broad applicability, GSLs hold great potential as cosmetic ingredients, as health foods or food supplements, and as therapeutics. However, their availability is limited since GSLs are characterized by a high structural complexity and their preparation represents a challenge.

Processes for the preparation of GSLs that are based on chemical and enzymatic synthesis exist.

Chemical synthesis is usually performed in two steps. First, the glycan moiety is synthesized and then coupled to ceramide or a sphingoid base. Drawbacks connected to this approach are the control of stereo- and regiochemistry, the need of multiple protecting group manipulations, difficult purification and scale-up.

Numerous attempts have been made to develop enzymatic methods for the production of GSLs. Enzymatic synthesis offers many advantages over purely chemical routes, such as high regio- and stereo-chemical control, it does not require the use of protecting group manipulations, and it is typically performed under mild conditions.

Glycosyltransferases (GT) have been used for the synthesis of GSLs. With this method the GSL sugar chain is constructed stepwise via the GT-catalyzed addition of constituent monosaccharides to a sphingoid base, a glycosylated sphingoid base, or a ceramide acceptor. Limitations to this approach include enzyme availability, the use of expensive glycosyl nucleotide donors, and the poor aqueous solubility of glycolipids.

Endoglycoceramidases (also termed herein EGCase, EC3.2.1.123) are a class of endonucleases belonging to glycoside hydrolase family 5 (GH5) which hydrolyze the glycoside linkage between the glycosyl moiety and the ceramide in glycosphingolipids.

Wildtype endoglycoceramidases typically have a conserved nucleophilic region including a conserved glutamate or aspartate. Endoglycoceramide synthases (also termed herein as “mutant endoglycoceramidases”, EGC synthases or EGCS), wherein the nucleophilic region has been mutated, especially the conserved glutamate or aspartate, has been exchanged to a non-nucleophilic residue, have been described in the art. It has been demonstrated that such mutant endoglycoceramidases have their hydrolytic activity reduced, while preserving the synthetic activity (see e. g. WO 2005/118798). The synthetic activity of the mutated endoglycoceramide synthases characterized to date is however usually not very strong, making an industrial scale-up challenging.

Enzymatic pathways suitable for the large-scale production of a wide variety of GSLs are lacking.

### SUMMARY OF THE INVENTION

The present invention relates to a polypeptide
a. comprising an amino acid motif of formula (1):

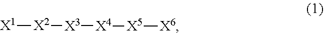

wherein
X¹ is an amino acid residue selected from I, M, L, V, A, F or W;
X² is an amino acid residue selected from L, M, I, V or A;
X³ is an amino acid residue A, L or M;
X⁴ is an amino acid residue selected from G, A, S, N, Q, C, T, I, V, L or M;
X⁵ is an amino acid residue selected from F, T, M, L or S;
X⁶ is an amino acid residue selected from G, L or F;
and
b. having glycosynthase activity.

The invention also relates to an isolated nucleic acid comprising a nucleic acid sequence encoding the polypeptide and genetically modified cells comprising said nucleic acid.

Other aspects of the invention relate to
the use of the polypeptide for the production of a glycosphingolipid;
a method of synthesizing a glycosphingolipid, the method comprising a step of reacting a glycosyl donor with a sphingolipid acceptor in the presence of the polypeptide of the invention.

Also, the present invention relates to a compound of formula (9), or a salt thereof:

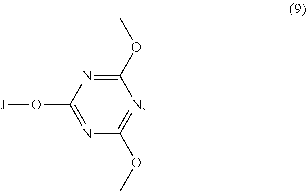

wherein

J is a glycosyl moiety selected from the group consisting of:

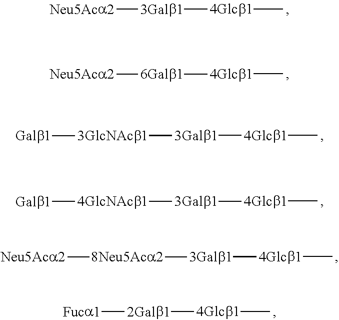

### DESCRIPTION OF FIGURES

FIGS. 1A and 1B show an overview of the SEQ ID NOs of the present invention.

FIG. 2 shows different acceptors that can be converted to glycosphingolipids by an endoglycoceramide synthase (EGCS) of the present invention (e.g. SEQ ID NO: 3, SEQ ID NO: 6 or SEQ ID NO: 8). A: ceramide N (18) S is used as acceptor; B: phytosphingosine is used as acceptor, C: dihydrosphingosine is used as acceptor.

FIG. 3 shows conversion of sphingosine to LNnT-sphingosine (LNnT-Sph) using an enzyme of SEQ ID NO: 6 and of SEQ ID NO: 8.

FIG. 4 shows conversion of sphingosine to 3′SL-sphingosine (3′SL-Sph) using an enzyme of SEQ ID NO: 8 at different temperatures, at 37° C. vs. at 22° C. (RT).

FIG. 5 shows conversion of sphingosine to LNT-sphingosine using an enzyme of SEQ ID NO: 6 and of SEQ ID NO: 8 and as a comparison of SEQ ID NO: 17.

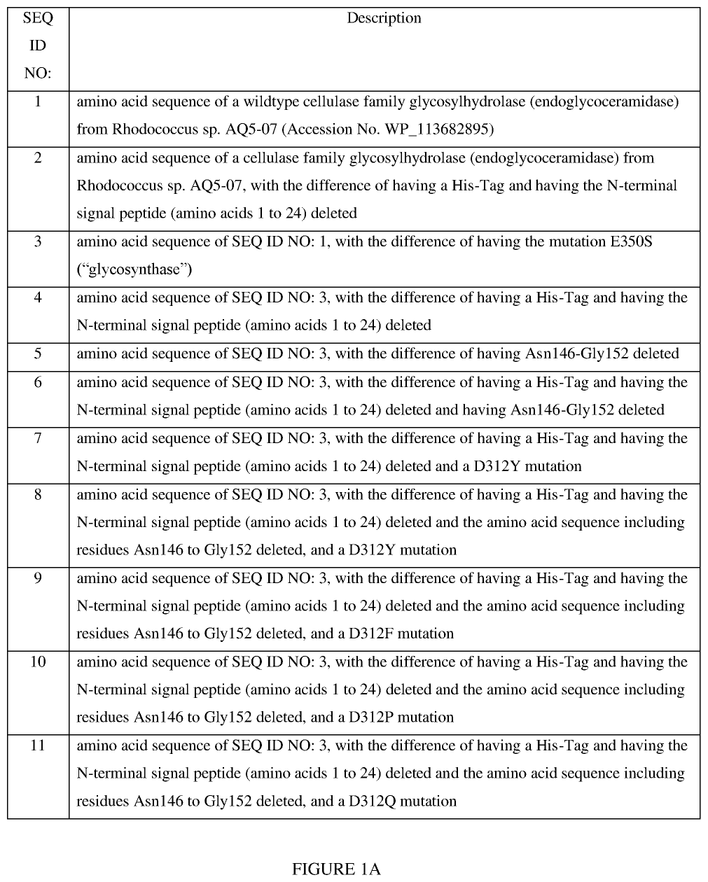

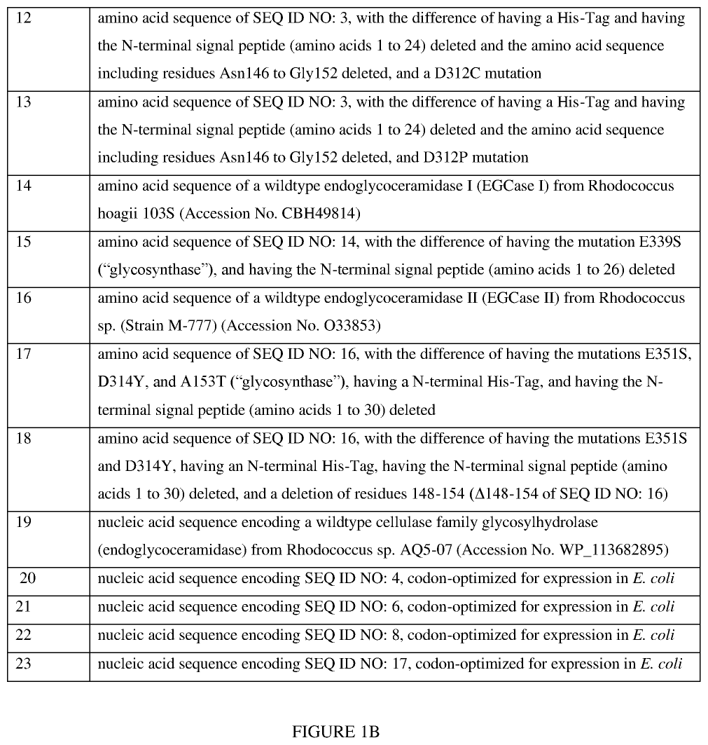

### DETAILED DESCRIPTION OF THE INVENTION

The present invention relates to novel recombinant polypeptides having glycosynthase enzymatic activity. The polypeptides of the invention are characterized by a high level of expression, high solubility and have a surprisingly high enzymatic activity and catalytic efficiency and are therefore suitable for use in both biocatalytic and biotechnological large-scale production of glycolipids, especially glycosphingolipids.

In particular, a first aspect of the present invention discloses a polypeptide
a. comprising an amino acid motif of formula (1)

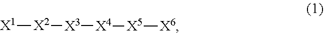

wherein
X¹ is an amino acid residue selected from I, M, L, V, A, F or W;
X² is an amino acid residue selected from L, M, I, V or A;
X³ is an amino acid residue A, L or M;
X⁴ is an amino acid residue selected from G, A, S, N, Q, C, T, I, V, L
or M;
X⁵ is an amino acid residue selected from F, T, M, L or S;
X⁶ is an amino acid residue selected from G, L or F;
and
b. having glycosynthase activity.

Non-limiting embodiments of different aspects of the invention are described below and illustrated by non-limiting examples.

The terms, definitions and embodiments described throughout the specification of the invention relate to all aspects and embodiments of the invention, unless mentioned otherwise.

As used herein, the term “comprising” or “comprises” is inclusive and does not exclude additional, unrecited elements, ingredients, or method steps. The phrase “consisting of” or “consists of” is closed and excludes any element, step, or ingredient not specified; and the phrase “consisting essentially of” or “consists essentially” means that specific further components can be present, namely those not materially affecting the essential characteristics of the compound, composition, or method. When used in the context of a sequence, the phrase “consisting essentially of” or “consists essentially” means that the sequence can comprise substitutions and/or additional sequences that do not change the essential function or properties of the sequence.

As used herein, the term “alkyl” refers to an acyclic straight or branched hydrocarbyl group having 1-50 carbon atoms which may be saturated or contain one or more double and/or triple bonds (so, forming for example an alkenyl or an alkynyl), and/or which may be substituted or unsubstituted, as herein further described. Examples of “alkyl” include, but are not limited to, methyl, ethyl, n-propyl, isopropyl, isobutyl, n-butyl, sec-butyl, tert-butyl, isopentyl, n-pentyl, neo-pentyl, n-hexyl, ethenyl, propenyl, 1-butenyl, 2-butenyl, isobutenyl, 1-pentenyl, 2-pentenyl, 2-methyl-1-butenyl, 3-methyl-1-butenyl, 2-methyl-2-butenyl, 1-hexenyl, 2-hexenyl, 3-hexenyl, methylpentenyl, dimethylbutenyl, ethynyl, propynyl, 1-butynyl, 2-butynyl, pentynyl, and hexynyl, each of which may be substituted or unsubstituted. Typically, the term alkyl refers to a straight saturated acyclic hydrocarbyl group having 1-31 carbons, which may be substituted or unsubstituted. The carbon chain length or range may be indicated, e.g. a C₁₋₃ alkyl refers to an alkyl having 1 to 3 carbons.

As used herein, the term “aryl” refers to an aromatic cyclic hydrocarbyl group having 5-14 ring carbon atoms, which may be mono- or polycyclic, which may contain fused rings, preferably 1 to 3 fused or unfused rings, and which may contain one or more heteroatoms, and/or which may be substituted or unsubstituted, as herein further described. Examples of “aryl” include, but are not limited to, phenyl, naphtyl, anthracyl, phenantryl, pyrrolyl, imidazolyl, thiophenyl, furanyl, oxazolyl, thiazolyl, pyridinyl, pyrimidinyl, pyrazinyl, triazinyl, and benzofuranyl, each of which may be substitute or unsubstituted. Typically, the term “aryl” refers to a substituted or unsubstituted phenyl.

As used herein, the term “acyl” refers to a group derived by the removal of one or more hydroxyl group from an oxoacid, preferably from a carboxylic acid. The acyl group according to the present invention is typically a saturated or unsaturated C₂₋₃₂ acyl, which may be substituted or unsubstituted.

As used herein, the term “substituted” means that the group in question is substituted with a group which typically modifies the general chemical characteristics of the group in question. The substituents can be used to modify characteristics of the molecule, such as molecule stability, molecule solubility and the ability of the molecule to form crystals. The person skilled in the art will be aware of other suitable substituents of a similar size and charge characteristics, which could be used as alternatives in a given situation.

In connection with the terms “alkyl”, “aryl”, and “acyl” the term substituted means that the group in question is substituted one or several times, preferably 1 to 3 times, with group(s) selected from hydroxy (which when bound to an unsaturated carbon atom may be present in the tautomeric keto form), oxo, C₁₋₆-alkoxy (i.e. C₁₋₆-alkyl-oxy), C₂₋₆-alkenyloxy, carboxy, oxo, C₁₋₆-alkoxycarbonyl, C₁₋₆-alkylcarbonyl, formyl, aryl, aryloxycarbonyl, aryloxy, arylamino, arylcarbonyl, heteroaryl, heteroarylamino, heteroaryloxycarbonyl, heteroaryloxy, heteroarylcarbonyl, amino, mono- and di(C₁₋₆-alkyl)amino, carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, cyano, guanidino, carbamido, C₁₋₆-alkyl-sulphonyl-amino, aryl-sulphonyl-amino, heteroaryl-sulphonyl-amino, C₁₋₆-alkanoyloxy, C₁₋₆-alkyl-sulphonyl, C₁₋₆-alkyl-sulphinyl, C₁₋₆-alkylsulphonyloxy, nitro, C₁₋₆-alkylthio, halogen, where any alkyl, alkoxy, and the like representing substituents may be substituted with hydroxy, C₁₋₆-alkoxy, C₂₋₆-alkenyloxy, carboxy, C₁₋₆-alkylcarbonylamino, halogen, C₁₋₆-alkylthio, C₁₋₆-alkyl-sulphonyl-amino, or guanidino.

The skilled person will understand that in formulas showing a specific compound, like for example formulas (4), (5), (8) and (9), unless the chemical formula expressly describes a carbon atom having a particular stereochemical configuration, the formula is intended to cover compounds where such a stereocenter has an R or an S configuration, or wherein a double bond has a cis or a trans configuration.

In the context of the present invention, the terms “about”, “around”, or “approximate” are applied interchangeably to a particular value (e.g. “a pH of about 4.5”, “a pH around 4.5”, or “a pH of approximate 4.5”), or to a range (e.g. “an amount from about 1% to about 99%”, “an amount from around 1% to around 99%”, or “an amount from approximate 1% to 30 approximate 99%”), to indicate a deviation from 0.1% to 10% of that particular value or range.

The term “cyclic structures” refers to a carbocycle ring, wherein all the ring atoms are carbons, or to a heterocycle ring, wherein one or more carbon atoms are replaced by an oxygen atom, a nitrogen atom and/or a sulfur atom. The carbocycle or the heterocycle cyclic structures are characterized by 5 to 8 ring atoms, preferably 5 to 6 ring atoms, may be saturated or contain double bonds, may be non-aromatic or aromatic and may be unsubstituted or substituted. Typically, cyclic structures are protecting groups, more preferably a phthaloyl protecting group, a tetrachlorophthaloyl protecting group or a vinylogous amide-type protecting group.

As used herein, the term “polypeptide” refers to a polymer of amino acids, and not to a specific length; thus, peptides, oligopeptides and proteins are included within the definition of a polypeptide.

The term “sphingolipid”, as used herein, refers to aliphatic amino alcohols such as sphingoid bases or analogs thereof (e.g. D-erythro-sphingosine, 6-hydroxy-D-erythro-sphingosine, D-ribo-phytosphingosine, DL-erythro-dihydrosphingosine), and ceramides or analogs thereof.

The term “leaving group”, as used herein, means an atom or a group (which may be charged or uncharged) that becomes detached from an atom belonging to the residual or main part of the molecule taking part in a specific reaction, such as for example a nucleophilic substitution or an elimination reaction.

As used herein, the term “glycosyl moiety” refers to a moiety deriving from a monosaccharide or from an oligosaccharide (more than one monosaccharide units). A glycosyl moiety deriving from an oligosaccharide unit may be linear or a branched. The monosaccharide unit can be any C₅₋₉ sugar, comprising aldoses (e.g. D-glucose, D-galactose, D-mannose, D-ribose, D-arabinose, L-arabinose, D-xylose, etc.), ketoses (e.g. D-fructose, D-sorbose, D-tagatose, etc.), deoxysugars (e.g. L-rhamnose, L-fucose, etc.), deoxy-aminosugars (e.g. N-acetylglucosamine, N-acetylmannosamine, N-acetylgalactosamine, etc.), uronic acids, ketoaldonic acids (e.g. sialic acid). The monosaccharide unit can form different cyclic structures such as pyranose (six-membered) cyclic structures or furanose (five-membered) cyclic structures.

The glycosyl moieties according to the present invention may be illustrated in the following style: Galβ1-4Glc1-, wherein the dash (-) represents the point of attachment of the glycosyl moiety and wherein the glycosyl moiety may be linked via an alpha or a beta glycosidic bond.

The term, “oligosaccharide portion of a ganglioside” as used herein is defined to encompass glycosyl moieties deriving from gangliosides, wherein the anomeric carbon at the reducing end of the oligosaccharide portion of the ganglioside is engaged in a glycosidic bond with another chemical entity, the glycosidic bond may be an alpha or a beta glycosidic bond, preferably a beta glycosidic bond. In the context of the present invention the terms oligosaccharide portion and glycosyl moiety may be used interchangeably.

The term “glycosphingolipid”, as used herein, refers to an O-glycoside wherein the aglycone moiety is a sphingolipid moiety, or an analogue thereof. The sphingolipid moiety may be composed of a sphingoid base moiety, or it may be composed of a ceramide moiety. Glycosphingolipids wherein the sphingolipid moiety is composed of a sphingoid base moiety may be referred to as glycosylated sphingoid bases. Glycosphingolipids wherein the sphingolipid moiety is a ceramide may be referred to as glycosylated ceramides.

In glycosphingolipids the sugar moiety is linked via a glycosidic bond with the hydroxyl group at the C-1 position of the sphingolipid moiety. The glycosidic linkage between the sphingolipid moiety and the glycosyl moiety may be an alpha (α), or a beta glycosidic (β) linkage. Preferably, the glycosidic linkage between the ceramide moiety and the glycosyl moiety is a β glycosidic linkage.

The term “cyclodextrin”, in the context of the present invention, refers to a cyclic oligosaccharide consisting of a macrocyclic ring of monosaccharide subunits (e.g., glucose). Cyclodextrins, typically contain 6-, 7- or 8-monosaccharide subunits and may be referred to as α-cyclodextrins, β-cyclodextrins, and γ-cyclodextrins, respectively. The cyclodextrin may be modified such that some or all of the primary or secondary hydroxyl groups of the macrocycle, or both, may be alkylated or acylated. Methods of modifying these alcohols are well known to the person skilled in the art and many derivatives are commercially available. Thus, some or all of the hydroxyl groups of the cyclodextrin may be substituted with an —OR¹⁰ group and/or an O—C(═O)—R¹¹ group, wherein R¹⁰ and R¹¹ are independently selected from a saturated or unsaturated C1-6 alkyl, a saturated or unsaturated C1-6 heteroalkyl, a saturated or unsaturated cycloalkyl, a saturated or unsaturated heterocycloalkyl, an aryl, or a heteroaryl, each of which may be substituted or unsubstituted. In some embodiments, R¹⁰ and R¹¹ are independently selected from the group consisting of 2-hydroxyethyl, 2-hydroxypropyl, and sulfobutylether.

The polypeptides of the invention have glycosynthase enzymatic activity. In a preferred embodiment, polypeptides have endoglycoceramide synthase enzymatic activity.

The term “glycosynthase” in the context of the present invention denotes an engineered glycosidase enzyme (also termed “glycoside hydrolase”), in which the catalytic nucleophile residue has been modified into a non-nucleophile residue, so that the hydrolytic activity of the enzyme is reduced. Typically, the nucleophilic residue, e. g. glutamate or aspartate, has been mutated. Further mutations may also be present, provided that the enzyme retains its synthetic activity unimpaired, or not significantly impaired. Accordingly, glycosynthases in the context of the present invention are mutant glycosidases which are hydrolytically impaired. The reduction of the hydrolytic activity may be e.g. around 50%, 60%, 70%, 80% or preferably around 90% or more. The term “around” as used herein means a deviation from the indicated value by 0.1-5%. The skilled person will know how to identify and replace these catalytic nucleophiles in the glycosidases (see e.g. Ly and Withers (1999) Annu. Rev. Biochem. 68, 487-522).

In connection with the term enzyme the term “functional analogue” refers to a protein wherein the amino acid sequence has a certain percent homology compared to the amino acid sequence of a reference protein (i.e. about 30% homology, preferably about 35%, 40%, 45%, 50%, 55%, 60%, 65%, 70%, 75%, 80%, 85%, 90%, 91%, 92%, 93%, 94%, 95%, 96%, 97%, 98%, 99% or higher homology over a specified region, for example over a region of at least about 25, 50, 75, 100, 150, 200, 250, 500, 1000, or more amino acids, up to the full length sequence, when compared and aligned for maximum correspondence over a comparison window or designated region) and maintains the same or similar functional activity of the reference protein. The percent homology may be determined using e.g. a BLAST sequence comparison algorithm, or by manual alignment and visual inspection (see e.g. NCBI website http://www.ncbi.nlm.nih.gov/BLAST/or the like). Such sequences may be termed “substantially identical”. Typically, the term functional analogue refers to a mutant protein, a truncated variant of the protein, or to a fusion protein which maintains the same functional activity of the reference protein.

A functional analogue of a glycosynthase is therefore an enzyme or polypeptide having glycosynthase enzymatic activity.

The glycosynthase of the present invention, as described herein, shows surprisingly improved properties compared to the described in the prior art, both solubility and synthetic activity.

Preferably, in one embodiment, the glycosynthase polypeptide comprises the amino acid motif X¹-X²-X³-X⁴-X⁵-X⁶ (1), wherein
X¹ is amino acid residue I;
X² is amino acid residue L;
X³ is amino acid residue A, L or M;
X⁴ is amino acid residue G, S or T;
X⁵ is amino acid residue F;
X⁶ is amino acid residue G.

In another preferred embodiment, the glycosynthase polypeptide comprises the amino acid motif X¹-X²-X³-X⁴-X⁵-X⁶ (1), wherein
X¹ is amino acid residue I;
X² is amino acid residue L;
X³ is amino acid residue A;
X⁴ is amino acid residue S or T;
X⁵ is amino acid residue F;
X⁶ is amino acid residue G.

In another preferred embodiment, the glycosynthase polypeptide comprises the amino acid motif X¹-X²-X³-X⁴-X⁵-X⁶ (1), wherein
X¹ is amino acid residue I;
X² is amino acid residue L;
X³ is amino acid residue A, L or M;
X⁴ is amino acid residue S;
X⁵ is amino acid residue F;
X⁶ is amino acid residue G.

In another preferred embodiment, the glycosynthase polypeptide comprises the amino acid motif X¹-X²-X³-X⁴-X⁵-X⁶ (1), wherein
X¹ is amino acid residue I;
X² is amino acid residue L;
X³ is amino acid residue A;
X⁴ is amino acid residue S;
X⁵ is amino acid residue F;
X⁶ is amino acid residue G.

Amino acid sequences are herein typically defined by the commonly used one-letter code or by their three-letter code, as summarized in Table 1.

TABLE 1
Amino acid codes:

| | | |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

A variant glycosynthase polypeptide or a fragment of a glycosynthase as used herein is a polypeptide having the glycosynthase functionality and comprising the amino acid motif X¹-X²-X³-X⁴-X⁵-X⁶ (1), wherein
X¹ is an amino acid residue selected from I, M, L, V, A, F or W;
X² is an amino acid residue selected from L, M, I, V or A;
X³ is an amino acid residue A, L or M;
X⁴ is an amino acid residue selected from G, A, S, N, Q, C, T, I, V, L or M;
X⁵ is an amino acid residue selected from F, T, M, L or S;
X⁶ is an amino acid residue selected from G, L or F. The variant polypeptide is usually an amino acid sequence having at least 70% sequence identity with the sequence of SEQ ID NO: 3, preferably an amino acid sequence having at least 80% sequence identity with the sequence of SEQ ID NO: 3, more preferably an amino acid sequence having at least 85% sequence identity with the sequence of SEQ ID NO: 3, even more preferably an amino acid sequence having at least 90% sequence identity with the sequence of SEQ ID NO: 3, especially an amino acid sequence having at least 95% sequence identity with the sequence of SEQ ID NO: 3.

In a preferred embodiment, the variant polypeptide is an amino acid sequence having at least 85% sequence identity with the sequence of SEQ ID NO: 3.

In a preferred embodiment, the variant polypeptide and/or fragment has an amino acid sequence in which amino acids Asn146 to Gly152 corresponding to SEQ ID NO: 3 have been deleted.

In a preferred embodiment, the variant polypeptide and/or fragment has a mutation on position D312 corresponding to SEQ ID NO: 3, wherein the mutation is preferably D312Y, D312F, D312C, D312P or D312Q.

In a preferred embodiment, the fragment of SEQ ID NO: 3 is lacking the N-terminal signal peptide sequence.

According to the invention, the variant polypeptide, e.g. as any of the above described, are functionally active as glycosynthase are capable of catalyzing the transfer of a glycosyl moiety from a glycosyl donor to a sphingolipid acceptor. The catalytically active polypeptides of the present invention can advantageously be used in a method of synthesizing a glycosphingolipid.

As used herein, the term “glycosyl donor” refers to a glycoside capable of reacting with a suitable “acceptor” molecule to form a new glycosidic bond. The resulting reaction is referred to as a glycosylation reaction, wherein a glycosyl moiety is transferred from the glycosyl donor to the acceptor molecule. Glycosylation reactions may be performed chemically, enzymatically in vitro, or enzymatically in vivo. A glycosylation reaction denotes in the context of the present invention an in vitro or an in vivo enzymatic glycosylation, wherein an enzyme having glycosynthase activity catalyzes the transfer of a glycosyl moiety from a suitable glycosyl donor to an acceptor molecule. Glycosyl donors suitable for use in the present invention typically possess a leaving group at the anomeric position which, upon activation, is eliminated forming an electrophilic anomeric carbon. Glycosyl donors suitable for use in the context of the present invention are typically α-glycosyl donors.

In the context of the present invention, the glycosyl donor is preferably a glycosyl donor of formula (2):

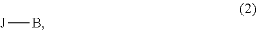

wherein
J is a glycosyl moiety,
B is selected from a fluoride, chloride, bromide, azide, formate, iodide.

The glycosyl donor is more preferably a glycosyl fluoride or a glycosyl chloride, even more preferably an α-glycosyl fluoride or an α-glycosyl chloride, especially an α-glycosyl fluoride.

The term, “acceptor”, when used herein, refers to a molecule containing an available nucleophile (e.g., an available hydroxyl group) that will react with a glycosyl donor to form a new glycosidic bond. Typically, the new glycosidic bond is formed via the nucleophilic attack of the available nucleophile of the acceptor to the electrophilic anomeric carbon of the donor. The resulting reaction is referred to as a glycosylation reaction, wherein a glycosyl moiety is transferred from the glycosyl donor to the acceptor molecule. Glycosylation reactions may be performed chemically, enzymatically in vitro, or enzymatically in vivo. A glycosylation reaction denotes in the context of the present invention an in vitro or an in vivo enzymatic glycosylation, wherein an enzyme having glycosynthase activity catalyzes the transfer of a glycosyl moiety from a suitable glycosyl donor to a suitable acceptor molecule. Suitable acceptors for use in the context of the present invention are typically sphingolipid acceptors. Sphingolipid acceptors are preferably represented by a compound of formula (3), or a salt thereof:

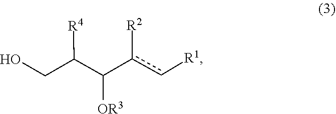

wherein
R¹ is H, aryl, or a C₁₋₂₀ alkyl, which may be saturated or contain one or more double and/or triple bonds, and/or which may contain one or more functional groups, the functional group being preferably selected from the group consisting of a hydroxyl group, an alkoxy group, an acyloxy group, a primary, secondary, or tertiary amine, an acylamido group, a thiol, a thioether or a phosphorus-containing functional group;
R² is H or —OR⁵, wherein R⁵ is selected from hydrogen, a substituted or unsubstituted C₁₋₃ alkyl, or a substituted or unsubstituted C₂₋₄ acyl;
the bond [symbol] may be a double or a single bond when R² is H, or is a single bond when R² is —OR⁵;
R³ is H, a substituted or unsubstituted C₁₋₃ alkyl, or a substituted or unsubstituted C₂₋₄ acyl;
R⁴ is N₃ or NR⁶R⁷, wherein R⁶ and R⁷ are independently selected from H, a substituted or unsubstituted C₂₋₃₂ acyl, a substituted or unsubstituted aryl, a substituted or unsubstituted vinyl, or wherein R⁶ and R⁷ form a cyclic structure.

Preferably, R¹ of (3) is a C₁₋₂₀ alkyl, more preferably R¹ of (3) is a C₁₃ alkyl.

In some embodiments, the sphingolipid acceptor is a compound of formula (8), or a salt thereof:

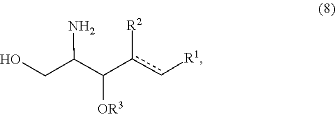

wherein
R¹, R², R³, and the bond [symbol] are as defined as for the compound of formula (3).

In some embodiments, when the sphingolipid acceptor is a compound of formula (8), the method further comprises an N-acylation of the sphingolipid moiety with a fatty acid. The N-acylation step may be performed enzymatically or chemically. In the N-acylation step, the addition of a fatty acid moiety is typically catalyzed, and wherein the fatty acid moiety is especially selected from a non-hydroxy fatty acid, an alpha-hydroxy fatty acid, and an omega-linoleoyloxy fatty acid.

In some preferred embodiments, the sphingolipid acceptor is a sphingoid base, especially D-erythro-sphingosine, 6-hydroxy-D-erythro-sphingosine, d-ribo-phytosphingosine, D-erythro-sphinganine.
“D-erythro-sphingosine” may also herein be referred to as “sphingosine”.
“D-ribo-phytosphingosine” may also herein be referred to as “phytosphingosine”.
“6-hydroxy-D-erythro-sphingosine” may also herein be referred to as “6-hydroxy-sphingosine”.
“D-erythro-sphinganine” may also herein be referred to as “dihydrosphingosine”.

In some preferred embodiments, the sphingolipid acceptor is a ceramide, especially CER[NS], CER[AS], CER[EOS], CER[NH], CER[AH], CER[EOH], CER[NP], CER[AP], CER[EOP], CER[NDS], CER[ADS] or CER[EODS].

The skilled person will understand that “CER” denotes ceramide. The following letters in [brackets] refer to the fatty acid groups of the ceramides, more precisely non-hydroxy fatty acids [N], alpha-hydroxy fatty acids [A], and omega-linoleoyloxy fatty acids [EO], and to the sphingoid base moieties of the ceramides, i.e. to sphingosine [S], phytosphingosine [P] and dihydrosphingosine [dS], respectively, according to the shorthand nomenclature developed by Motta et al. (1993) Biochim Biophys Acta. 1182:147-151 and expanded by Rabionet (2014) Biochim Biophys Acta. 1841:422-434. For each species, the number of carbons and unsaturations (if present) may be expressed in parentheses following the letters of N, A, E, and O.

The term “glycoside” when used herein refers to a chemical compound wherein a glycosyl moiety is bound to non-sugar chemical moiety via a glycosidic linkage. Glycosides can be linked by an O- (an O-glycoside), N- (a glycosylamine), S- (a thioglycoside), or C- (a C-glycoside) glycosidic linkage. The sugar moiety is typically referred to as “glycone”, and the non-sugar chemical moiety is typically referred to as the “aglycone”. The “glycone” may consist of a single sugar moiety (monosaccharide), two sugar groups (disaccharide), or several sugar groups (oligosaccharide).

In the context of the present invention, a compound of formula (3) represents a “glycoside”, wherein the glycosyl moiety (glycone) J is bound via a glycosidic linkage to the aglycone R¹². The glycosidic linkage may be an alpha (α) or a beta (β) glycosidic linkage. Preferably, the aglycone R¹² is bound to the glycosyl moiety (glycone) J via a β glycosidic linkage.

In some embodiments, the glycosyl donor is generated in-situ from a glycoside of formula (4), or a salt thereof:

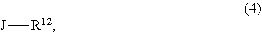

wherein
J is as defined as for the glycosyl donor of formula (2);
R¹² is selected from a 4,6-disubstituted 1,3,5-triazyn-2-yloxy group, a substituted or unsubstituted dinitrophenyloxy group, a substituted or unsubstituted dimedonyloxy group and a pentafluorophenyloxy group.

In some embodiments, R¹² of the glycoside of formula (4) is a 4,6-disubstituted 1,3,5-triazyn-2-yloxy group. Accordingly, in some embodiments, the glycoside of formula (4) is a glycoside of formula (5), or a salt thereof:

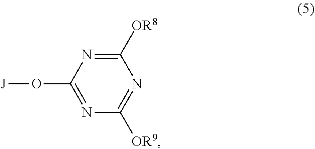

wherein
J is as defined as for the glycosyl donor of formula (2);
R⁸ and R⁹ are independently selected form the group consisting of methyl, ethyl, benzyl, 2,2,2-trifluoroethyl, preferably methyl.

In some embodiments, R¹² of the glycoside of formula (3) is a 4,6-dimethyl 1,3,5-triazyn-2-yloxy group. Accordingly, in some embodiments, the glycoside of formula (3) is a glycoside of formula (9), or a salt thereof:

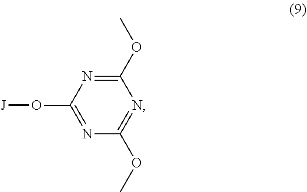

wherein

J is as defined as for the glycosyl donor of formula (2).

The in-situ generation of the glycosyl donor is typically performed via reacting the glycoside of formula (4), (5) or (9) with a nucleophile in the presence of the polypeptide according to the present invention.

The term “nucleophile”, as used herein generally refers to an ion or a molecule that donates a pair of electrons to an atomic nucleus to form a covalent bond.

Suitable nucleophiles for use in the present invention are conjugate bases of organic or inorganic acids. Suitable nucleophiles include, but are not limited to, the conjugate bases derived from hydrofluoric acid, hydrochloric acid, hydrobromic acid, hydroiodic acid, formic acid, and hydrazoic acid.

Conjugate bases derived from hydrofluoric acid, hydrochloric acid, hydrobromic acid, hydroiodic acid, formic acid, and hydrazoic acid may also be referred to as fluoride, chloride, bromide, iodide, formate, and azide, respectively.

The nucleophile may be added to the reaction mixture as an inorganic or an organic salt. Suitable salts include, but are not limited to, NaF, KF, NH₄F, MgF₂, CaF₂, NaHF₂, KHF₂, NH₄HF₂, NaCl, KCl, NH₄Cl, MgCl₂, CaCl₂), NaBr, KBr, NH₄Br, MgBr₂, CaBr₂, NaI, KI, NH₄I, MgI₂, CaI₂, HCOONa, HCOOK, HCOONH₄, (HCOO)₂Mg, (HCOO)₂Ca, NaN₃, KN₃, NH₄N₃, MgN₆, and CaN₆.

In some embodiments, the nucleophile is the conjugate base derived from hydrochloric acid. Accordingly in some embodiments the nucleophile is a chloride.

The chloride may be added to the reaction mixture as an inorganic or an organic salt. Suitable salts include, but are not limited to, NaCl, KCl, NH₄Cl, MgCl₂, CaCl₂, NaHCl₂, KHCl₂, NH₄HCl₂. Typically, the chloride is added as NaCl, or KCl.

In some preferred embodiments, the nucleophile is the conjugate base derived from hydrofluoric acid. Accordingly in some preferred embodiments the nucleophile is a fluoride. The fluoride may be added to the reaction mixture as an inorganic or an organic salt. Suitable salts include, but are not limited to, NaF, KF, NH₄F, MgF₂, CaF₂, NaHF₂, KHF₂, NH₄HF₂.

Typically, the fluoride is added as KF, or KHF₂.

In some embodiments, the present invention relates to a method of synthesizing a glycosphingolipid, the method comprising reacting a glycosyl donor, such as a glycosyl donor of formula (2), with a sphingolipid acceptor, such as a sphingolipid acceptor formula (4) or (8), in the presence of a polypeptide according to the present invention.

The method or the glycosylation reaction step of the method may be performed at a temperature selected from the range of between 15° C. and 40° C. For example, the method or the glycosylation reaction step of the method may be performed at 37° C.

Suitable polypeptides according to the present invention are polypeptides having glycosynthase enzymatic activity. The polypeptide may be any glycosynthase sequence which has yet to be determined. Glycosynthases yet to be determined can be identified using sequence databases and sequence alignment algorithms, for example, the publicly available GenBank database and the BLAST alignment algorithm.

In a preferred embodiment, the suitable polypeptides according to the present invention have endoglycoceramide synthase enzymatic activity. As described above a polypeptide having endoglycoceramide synthase enzymatic activity may also be referred to as endoglycoceramide synthase (EGC synthase), or mutant endoglycoceramidase.

In some embodiments, EGC synthases for use in the context of the present invention include but are not limited to those summarized in Table 2.

TABLE 2
Overview of EGC synthases

| | Mutation(s)/ | |
|---|---|---|
| | Modifications as | |
| EGC synthase | compared to wildtype | Source (wildtype sequence) |
| SEQ ID | E350S | Rhodococcus sp. AQ5-07 |
| NO: 3 | | (SEQ ID NO: 1) |
| SEQ ID | E350S, Δ1-24, | Rhodococcus sp. AQ5-07 |
| NO: 4 | N-term. His-Tag | (SEQ ID NO: 1) |
| SEQ ID | E350S, Δ146-152 | Rhodococcus sp. AQ5-07 |
| NO: 5 | | (SEQ ID NO: 1) |
| SEQ ID | E350S, Δ1-24, | Rhodococcus sp. AQ5-07 |
| NO: 6 | N-term. His-Tag, | (SEQ ID NO: 1) |
| | Δ146-152 | |
| SEQ ID | E350S, D312Y, Δ1-24, | Rhodococcus sp. AQ5-07 |
| NO: 7 | N-term. His-Tag | (SEQ ID NO: 1) |
| SEQ ID | E350S, D312Y, Δ1-24, | Rhodococcus sp. AQ5-07 |
| NO: 8 | N-term. His-Tag, | (SEQ ID NO: 1) |
| | Δ146-152 | |
| SEQ ID | E350S, D312F, Δ1-24, | Rhodococcus sp. AQ5-07 |
| NO: 9 | N-term. His-Tag, | (SEQ ID NO: 1) |
| | Δ146-152 | |
| SEQ ID | E350S, D312P, Δ1-24, | Rhodococcus sp. AQ5-07 |
| NO: 10 | N-term. His-Tag, | (SEQ ID NO: 1) |
| | Δ146-152 | |
| SEQ ID | E350S, D312Q, Δ1-24, | Rhodococcus sp. AQ5-07 |
| NO: 11 | N-term. His-Tag, | (SEQ ID NO: 1) |
| | Δ146-152 | |
| SEQ ID | E350S, D312C, Δ1-24, | Rhodococcus sp. AQ5-07 |
| NO: 12 | N-term. His-Tag, | (SEQ ID NO: 1) |
| | Δ146-152 | |
| SEQ ID | E350S, D312P, Δ1-24, | Rhodococcus sp. AQ5-07 |
| NO: 13 | N-term. His-Tag, | (SEQ ID NO: 1) |
| | Δ146-152 | |
| SEQ ID | E339S, Δ1-26 | Rhodococcus hoagii 103S |
| NO: 15 | | (SEQ ID NO: 14) |
| SEQ ID | E351S, D314Y, A153T, | Rhodococcus sp. |
| NO: 17 | Δ1-30, N-term. His-Tag | strain M-777 |
| | | (SEQ ID NO: 16) |
| SEQ ID | E351S, D314Y, Δ1-30, | Rhodococcus sp. |
| NO: 18 | N-term. His-Tag, Δ148-154 | strain M-777 |
| | | (SEQ ID NO: 16) |

In some embodiments, the EGC synthase for use in the context of the present invention is selected from the group consisting of EGC synthase SEQ ID NO: 3, EGC synthase SEQ ID NO: 4, EGC synthase SEQ ID NO: 5, EGC synthase SEQ ID NO: 6, EGC synthase SEQ ID 5 NO: 7, and EGC synthase SEQ ID NO: 8, EGC synthase SEQ ID NO: 9, EGC synthase SEQ ID NO: 10, EGC synthase SEQ ID NO: 11, EGC synthase SEQ ID NO: 12, and EGC synthase SEQ ID NO: 13.

In some preferred embodiments, the EGC synthases for use in the context of the present invention is EGC synthase of SEQ ID NO: 3 or of SEQ ID NO: 8.

10 The EGC synthases of the present invention may be provided as purified proteins or as cell-free extract, in freeze-dried form or in spray-dried form.

In some embodiments, the glycosyl donor is generated in-situ.

In some embodiments, the present invention describes a method for the production of a glycosphingolipid, the method comprising reacting a glycoside of (4), or a salt thereof:

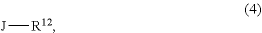

wherein
J is a glycosyl moiety,
R¹² is a selected from a 4,6-disubstituted 1,3,5-triazyn-2-yloxy group, a substituted or unsubstituted dinitrophenyloxy group, a substituted or unsubstituted dimedonyloxy group and a pentafluorophenyloxy group,
with a sphingolipid acceptor, and a nucleophile, wherein the nucleophile is selected from fluoride, chloride, bromide, azide, formate, or iodide, in the presence of a polypeptide according to the present invention;
and wherein the glycoside of formula (4) is converted into a glycosyl donor of formula (2):

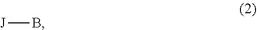

wherein
J is a glycosyl moiety,
B is selected from a fluoride, chloride, bromide, azide, formate, iodide;
thereby producing the glycosphingolipid.

In those embodiments, where the glycosyl donor is generated in-situ, the glycosyl donor is typically directly consumed during the production of the glycosphingolipid. Accordingly, the person skilled in the art would understand that typically, the glycosyl donor, when generated in-situ, does not accumulate nor can be isolated from the reaction mixture.

Typically, when the glycosyl donor is generated in-situ, the in-situ formation occurs via a nucleophilic substitution, wherein the group R¹² of the glycoside of formula (3) is replaced by the nucleophile. Typically, the nucleophilic substitution occurs in the presence of the polypeptide according to the present invention.

The group R¹² of the glycoside of formula (4) is preferably bound to the glycosyl moiety J via a beta glycosidic linkage whereas the group B of the glycosyl donor of formula (2) is preferably bound to the glycosyl moiety J via an alpha glycosidic linkage. Accordingly, the nucleophilic substitution results in a change of the stereochemical configuration of the glycosidic bond. The person skilled in the art would understand that the group R¹² of the glycoside of formula (3) and the group B of glycosyl donor of formula (2) may both represent leaving groups. Typically, however only the group B of the compound formula (2) may be activated as a leaving group under the conditions of the glycosylation method of the present invention. Accordingly, only the compound of formula (2) functions as a glycosyl donor in the context of the present invention.

In some embodiment, the glycosyl moiety J of the glycosyl donor of formula (2), and/or of the glycoside of formula (3) is Galβ1-4Glc-, and wherein the method further comprises the use of an enzyme having β-galactosidase activity.

In some embodiments, the present invention describes a method for the production of a glycosphingolipid, the method comprising the steps of:
reacting a glycosyl donor of formula (10):

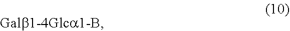

wherein
B is selected from a fluoride, chloride, bromide, azide, formate, iodide;
with a sphingolipid acceptor in the presence of a polypeptide according to the present invention, and sequentially
adding an enzyme having β-galactosidase activity to the mixture of the preceding step, thereby producing a glycosphingolipid of formula (11):

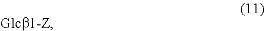

wherein Z is a sphingolipid moiety.

The term “an enzyme having a β-galactosidase activity” may be interchangeably used with the term “β-galactosidase” and denotes, in the context of the present invention, an enzyme belonging to the glycoside hydrolase family 35 (GH35) which typically catalyses the hydrolysis of terminal non-reducing β-D-galactose residues in β-D-galactosides.

In the context of the present invention a β-galactosidase may also be referred to as lactase.

The β-galactosidase in its wild-type form, may originate from microorganisms such as bacteria, yeasts, ascomycete, actinomycetes, hyphomycetes, basidiomycotina, and the like.

The β-galactosidase, in its wild-type form, may originate from Aspergillus oryzae.

The β-galactosidase in its wildtype form, may be recombinantly expressed by a host microorganism, either as plasmid-borne or genome integrated.

In general, any enzyme having β-galactosidase activity as defined above is suitable for the purpose of the invention. Accordingly, the enzyme may originate from any known β-galactosidase sequence or from any β-galactosidase sequence which has yet to be determined. β-Galactosidase yet to be determined can be identified using sequence databases and sequence alignment algorithms, for example, the publicly available GenBank database and the BLAST alignment algorithm.

In some embodiments, the enzyme having β-galactosidase activity is a wild-type β-galactosidase originating from Aspergillus oryzae, or a functional analogue thereof. The amino acid sequence of the wild-type β-galactosidase originating from Aspergillus oryzae can be found on https://www.uniprot.org/, the accession number: Q2UCU3.

In some preferred embodiments, the enzyme having β-galactosidase activity is a truncated variant of the wild-type β-galactosidase originating from Aspergillus oryzae (Q2UCU3). The truncated variant of the β-galactosidase, according to the present invention, can be purchased from established manufacturers, e.g. Calza Clemente, or produced by methods known to the skilled person such as that described in M. M. Maksimainen et al., International Journal of Biological Macromolecules 2013, 60, 109-115.

In some embodiments, the present invention describes a method for the production of a glycosphingolipid, the method comprising the steps of:
reacting a glycoside of (12), or a salt thereof:

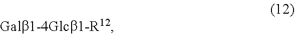

wherein
R¹² is a selected from a 4,6-disubstituted 1,3,5-triazyn-2-yloxy group, a substituted or unsubstituted dinitrophenyloxy group, a substituted or unsubstituted dimedonyloxy group and a pentafluorophenyloxy group,
with a sphingolipid acceptor, and a nucleophile, wherein the nucleophile is selected from fluoride, chloride, bromide, azide, formate, or iodide, in the presence of a polipepetide according to the present invention;
and wherein the glycoside of formula (12) is converted into a glycosyl donor of formula (10):

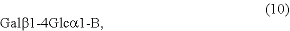

wherein
B is selected from a fluoride, chloride, bromide, azide, formate, iodide, and sequentially
adding an enzyme having β-galactosidase activity to the mixture of the preceding step, thereby producing a glycosphingolipid of formula (11):

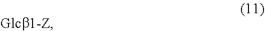

wherein Z is a sphingolipid moiety.

In some embodiments, the method further comprises the use of an enzyme having β-galactosidase activity in the presence of a cyclodextrin in the reaction mixture.

In some embodiments, the present invention describes a method for the production of a glycosphingolipid, the method comprising the steps of:
reacting a glycosyl donor of formula (10):

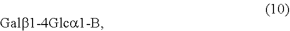

wherein
B is selected from a fluoride, chloride, bromide, azide, formate, iodide;
with a sphingolipid acceptor in the presence of a polypeptide according to the present invention, and sequentially
adding an enzyme having β-galactosidase activity, and a cyclodextrin to the mixture of the preceding step, thereby producing a glycosphingolipid of formula (11):

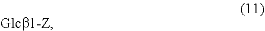

wherein Z is a sphingolipid moiety.

In some embodiments, the present invention describes a method for the production of a glycosphingolipid, the method comprising the steps of:
reacting a glycoside of (12), or a salt thereof:

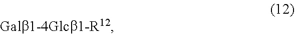

wherein
R¹² is a selected from a 4,6-disubstituted 1,3,5-triazyn-2-yloxy group, a substituted or unsubstituted dinitrophenyloxy group, a substituted or unsubstituted dimedonyloxy group and a pentafluorophenyloxy group,
with a sphingolipid acceptor, and a nucleophile, wherein the nucleophile is selected from fluoride, chloride, bromide, azide, formate, or iodide, in the presence of a polypeptide according to the present invention;
and wherein the glycoside of formula (12) is converted into a glycosyl donor of formula (10):

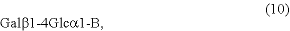

wherein
B is selected from a fluoride, chloride, bromide, azide, formate, iodide, and sequentially
adding an enzyme having β-galactosidase activity, and a cyclodextrin to the mixture of the preceding step, thereby producing a glycosphingolipid of formula (11):

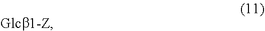

wherein Z is a sphingolipid moiety.

In some embodiments, the cyclodextrin is α-cyclodextrin, β-cyclodextrin, γ-cyclodextrin, or derivatives thereof.

In some embodiments, the cyclodextrin is selected from the group consisting of β-cyclodextrin, hydroxypropyl-β-cyclodextrin, randomly methylated β-cyclodextrin, or sulfobutylether-β-cyclodextrin. In some preferred embodiments, the cyclodextrin is β-cyclodextrin.

The cyclodextrin is typically used in an amount between about 0.1 equivalents to about 1 equivalent based on the amount of the glycosphingolipid. In some preferred embodiments the cyclodextrin is used in an amount between about 0.1 equivalents to about 0.5 equivalents based on the amount of the glycosphingolipid. Accordingly, in some preferred embodiments, the cyclodextrin is used in an amount of about 0.1, 0.2, 0.3, 0.4, or 0.5 equivalents based on the amount of the glycosphingolipid.

The use of a cyclodextrin provides several advantages such as high yields, it facilitates the purification of the resulting glycosphingolipid, and eliminates the need for the use of a detergent or organic solvent to increase accessibility to the glycosyl moiety of the substrate. However, detergents or organic solvents can also be used in the method of the invention.

The glycosyl donor or the glycoside, the sphingolipid acceptor, and the polypeptide according to the present invention may be combined by admixture in an aqueous reaction medium. The medium generally has a pH value of about 5 to about 7.5. The selection of the medium is based on the ability of the medium to maintain the pH value at the desired level. Accordingly, in some embodiments the medium is buffered to a pH value of about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5. In some preferred embodiments, the medium is buffered to a pH value of about 6.0 to 6.5. Accordingly, in some preferred embodiments, the medium is buffered to a pH value of 6.0, 6.1, 6.2, 6.3, 6.4, 6.5.

Suitable buffers include, but are not limited to, MES, Bis-Tris, ADA, ACES, PIPES, MOPSO, MOPS, HEPES, PBS, sodium acetate buffer, sodium citrate buffer. Preferably, sodium acetate buffer. If a buffer is not used, the pH of the medium should be maintained at about 5 to about 7.5.

The reaction medium may also comprise carboxylic acids, such as formic acid, acetic acid, propionic acid, glyceric acid, pyruvic acid, malonic acid, butanoic acid, fumaric acid, maleic acid, valeric acid, isovaleric acid, pivalic acid, glutaric acid, and caproic acid. Preferably, formic acid, acetic acid, propionic acid, valeric acid, or caproic acid. More preferably, acetic acid. Typically, the carboxylic acid is added at a concentration range of about 500 mM to 5 M, preferably 500 mM to 2 M, more preferably from 500 mM to 1 M.

In those embodiments, wherein a nucleophile is used, the nucleophile may be added to the reaction mixture as an inorganic or an organic salt. Suitable salts include, but are not limited to, NaF, KF, NH₄F, MgF₂, CaF₂, NaHF₂, KHF₂, NH₄HF₂, NaCl, KCl, NH₄Cl, MgCl₂, CaCl₂, NaBr, KBr, NH₄Br, MgBr₂, CaBr₂, NaI, KI, NH₄I, MgI₂, CaI₂, HCOONa, HCOOK, HCOONH₄, (HCOO)₂Mg, (HCOO)₂Ca, NaN₃, KN₃, NH₄N₃, MgN₆, and CaN₆. Preferably, NaCl, KCl, KF, NaF, or KHF₂.

The glycosyl donor or the glycoside, the sphingolipid acceptor, the polypeptide according to the present invention, as well as any other component used during the method of the present invention may be added to the reaction mixture either as a solid or dissolved in a solvent, and in any quantities and manner effective for the intended result of the process.

The reaction is allowed to proceed for a period of time sufficient to obtain the desired high yield of the desired glycosphingolipid.

Typically, the reaction is allowed to proceed for between about 30 minutes to about 24 hours, preferably between about 6 to about 48 hours, preferably between about 18 to about 24 hours.

In some embodiments, reaction is allowed to proceed for about 18, 19, 20, 21, 22, 23, or 24 hours.

In the context of the present invention enzymes or polypeptides amounts, or concentrations are typically expressed in activity units (U), which is the measure of initial rate of catalysis. One activity unit catalyses the formation of 1 μmol of product per minute at a given temperature and pH value.

The enzymes may be provided as purified proteins, as cell-free extract, or as lysate.

In some embodiments, the enzymes or polypeptides are provided as purified proteins, with a purity of about 50% to about 95%.

In some preferred embodiments, the enzymes or polypeptides are provided as cell-free extract, wherein the cell-free extract contains from about 5 wt % to about 70 wt % of the enzyme. Preferably, the cell-free extract contains from about 20 wt % to about 70 wt % of the enzyme.

In some embodiments the enzymes or polypeptides are provided as lysate, wherein the lysate contains from about 5 wt % to about 70 wt % of the specific enzyme. Preferably, the lysate contains from about 20 wt % to about 70 wt % of the specific enzyme.

The enzymes or polypeptides are usually present in a catalytic amount. The catalytic efficiency of a particular enzyme or polypeptide varies according to the concentration of that enzyme's substrate as well as to the reaction conditions such as temperature, time, and pH value. Means for determining the catalytic amount for a given enzyme under preselected substrate concentrations and reaction conditions are well known to those skilled in the art.

In some embodiments, the glycosyl donor is generated in-situ from a glycoside of formula (5), and wherein the method further comprising a step of synthesizing the glycoside of formula (5).

In some embodiments, the present invention relates to a method for the production of a glycosphingolipid, the method comprising the steps of:
reacting a saccharide of formula (6), or a salt thereof:

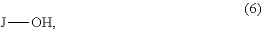

wherein
J is a glycosyl moiety,
with a compound of formula (7):

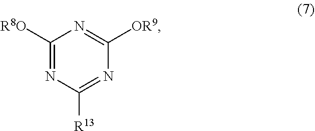

wherein R⁸ and R⁹ are as defined as for the glycoside of formula (5);
R¹³ is a halide selected from iodide, chloride, bromide, and fluoride, preferably chloride;
in the presence of an organic base and an inorganic base, and wherein the organic base is present in a catalytic amount, thereby producing a glycoside of formula (5), or a salt there of:

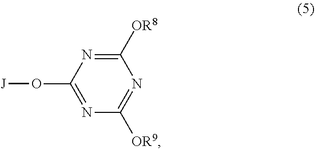

wherein
J is a glycosyl moiety;
R⁸ and R⁹ are independently selected form the group consisting of methyl, ethyl, benzyl, 2,2,2-trifluoroethyl, preferably methyl.
reacting a glycoside of (5), with a sphingolipid acceptor, and a nucleophile, wherein the nucleophile is selected from fluoride, chloride, bromide, azide, formate, or iodide, in the presence of a polypeptide according to the present invention;
and wherein the glycoside of formula (5) is converted into a glycosyl donor of formula (2):

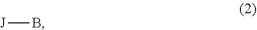

wherein
J is a glycosyl moiety,
B is selected from fluoride, chloride, bromide, azide, formate, iodide,
thereby producing the glycosphingolipid.

In some embodiments, the present invention relates to a method for the production of a glycosphingolipid, the method comprising the steps of:
reacting the saccharide Galβ1-4Glc-OH with a compound of formula (7):

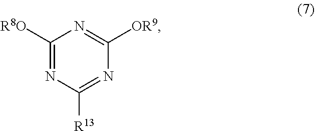

wherein R⁸ and R⁹ are as defined as for the glycoside of formula (5);
R¹³ is a halide selected from iodide, chloride, bromide, and fluoride, preferably chloride;
in the presence of an organic base and an inorganic base, and wherein the organic base is present in a catalytic amount, thereby producing a glycoside of formula (13):

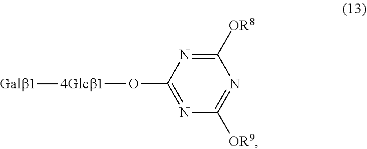

wherein
R⁸ and R⁹ are independently selected form the group consisting of methyl, ethyl, benzyl, 2,2,2-trifluoroethyl, preferably methyl;
reacting the glycoside of formula (13), with a sphingolipid acceptor, and a nucleophile, wherein the nucleophile is selected from a fluoride, chloride, bromide, azide, format, or iodide, in the presence of a polypeptide according to the present invention;
and wherein the glycoside of formula (13) is converted into a glycosyl donor of formula (10):

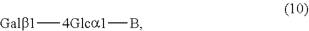

wherein
B is selected from a fluoride, chloride, bromide, azide, formate, iodide, and sequentially
adding an enzyme having β-galactosidase activity to the mixture of the preceding step, thereby producing a glycosphingolipid of formula (11):

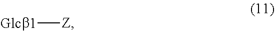

wherein Z is a sphingolipid moiety.

In some preferred embodiments, the glycoside of formula (5) is a glycoside of formula (9), or a salt thereof:

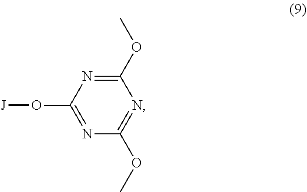

wherein J is a glycosyl moiety selected from the group consisting of the following glycosyl moiety, or salts thereof:

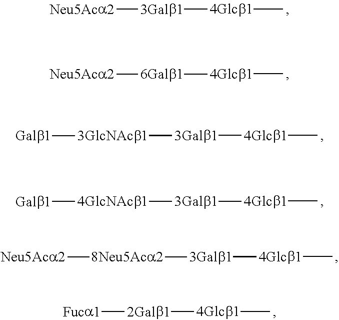

In some preferred embodiments, the 1,3,5-triazin-2-yl-glycosides is selected from the following compounds, or salts thereof:

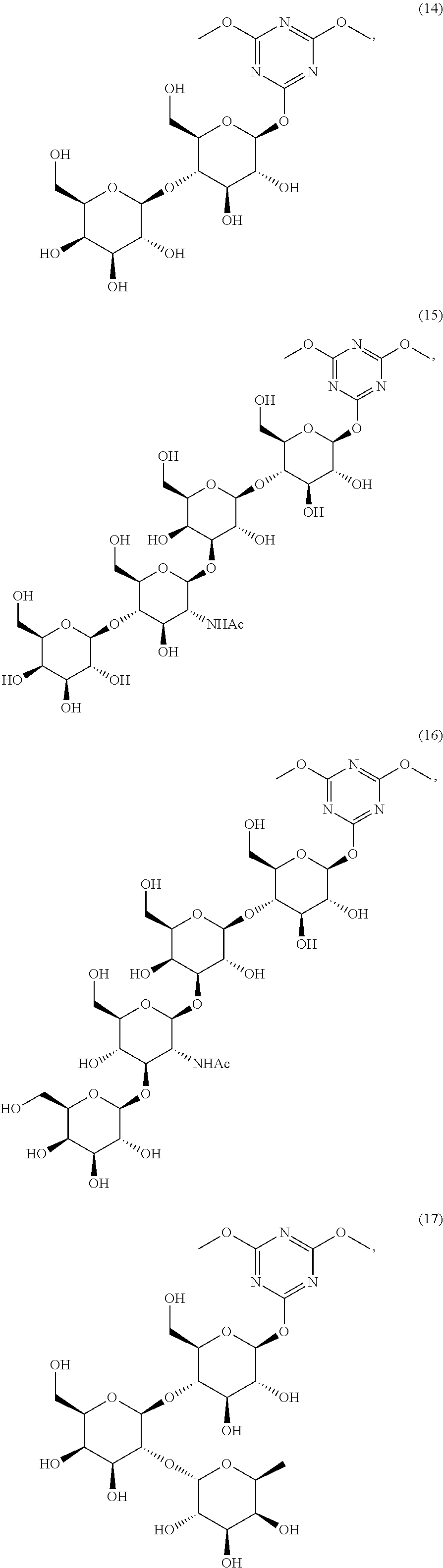

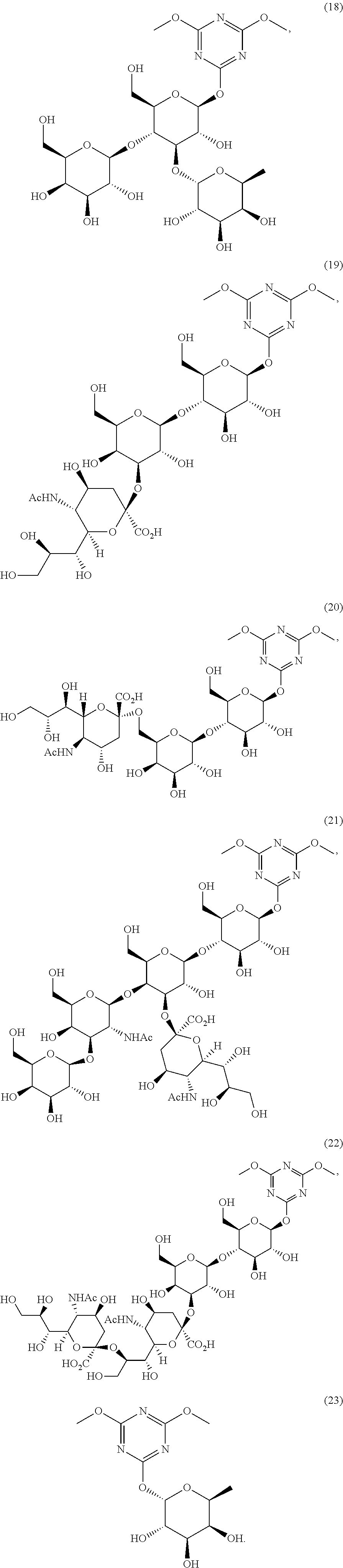

Typically, the reaction between the saccharide of formula (6) and the compound of formula (7) is performed in a solvent such as water, a mixture of water and an alcohol, or a mixture of water and acetonitrile.

In some preferred embodiments, the reaction between the saccharide of formula (6) and the compound of formula (7) is performed in water.

In some embodiments, the reaction between the saccharide of formula (6) and compound of formula (7) is performed in a mixture of water and an alcohol, such as a mixture of water and methanol, water and ethanol, or water and isopropanol, and wherein the alcohol constitutes from about 5% to about 15% of the mixture.

In some embodiments, the reaction between the saccharide of formula (6) and the compound of formula (7) is performed in a mixture of water and acetonitrile, wherein the acetonitrile constitutes from about 5% to about 15% of the mixture.

The inorganic base is typically a base such as NaHCO₃, Na₂CO₃, KHCO₃, K₂CO₃, (NH₄)₂CO₃, or ammonia, and it is typically present in an amount of about 1.3 to about 2.5 molar equivalents based on the amount of the saccharide. In some embodiments, the inorganic base is present in an amount of about 1.3, 1.4, 1.5, 1.6, 1.7, 1.8, 1.9, 2.0, 2.1, 2.2, 2.3, 2.4 or 2.5 molar equivalent based on the amount of the saccharide.

In some preferred embodiments, the inorganic base is NaHCO₃, wherein the NaHCO₃ is present in an amount of about 1.3, 1.4, 1.5, 1.6, 1.7, 1.8, 1.9, 2.0, 2.1, 2.2, 2.3, 2.4 or 2.5 molar equivalent based on the amount of the saccharide.

The organic base is typically a base such as 4-methylmorpholine, 1,4-diazabicyclo[2.2.2]octane, 1,8-diazabicycloundec-7-ene, 1,5-diazabicyclo(4.3.0) non-5-ene, 2,6-di-tert-butylpyridine, and it is typically present in a catalytic amount of about 0.05 to about 0.2 molar equivalents based on the amount of the saccharide. In some embodiments, the organic base is present in a catalytic amount of about 0.05, 0.06, 0.07, 0.08, 0.09, 0.10, 0.11, 0.12, 0.13, 0.14, 0.15, 0.16, 0.17, 0.18, 0.19, or 0.20 molar equivalents based on the amount of the saccharide.

In some preferred embodiments, the organic base is 4-methylmorpholine, wherein the 4-methylmorpholine is present in a catalytic amount of about 0.05, 0.06, 0.07, 0.08, 0.09, 0.10, 0.11, 0.12, 0.13, 0.14, 0.15, 0.16, 0.17, 0.18, 0.19, or 0.20 molar equivalents based on the amount of the saccharide.

Typically, the compound of formula (7) is present in an amount of about 1 to about 2 molar equivalents based on the amount of the saccharide. In some embodiments, the compound of (7) is present in an amount of about 1.0, 1.1, 1.2, 1.3, 1.4, 1.5, 1.6, 1.7, 1.8, 1.9, or 2.0 molar equivalents based on the amount of the saccharide.

In some preferred embodiments, the compound of formula (7) is 2-chloro-4,6-dimethoxy-1,3,5-triazine, wherein the 2-chloro-4,6-dimethoxy-1,3,5-triazine is present in an amount of about 1.0, 1.1, 1.2, 1.3, 1.4, 1.5, 1.6, 1.7, 1.8, 1.9, or 2.0 molar equivalents based on the amount of the saccharide.

Typically, the reaction between the saccharide and the compound of formula (7) is performed at a temperature of about 0° C. to about 25° C. In some embodiments, the reaction is performed at a temperature of about 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., or 25° C.

Glycosyl donors, sphingolipids acceptors, and glycosides according to the present invention, may be produced by methods known to the skilled person.

A method for the synthesis of glycosyl donors such as a glycosyl fluoride donors is, for example, described in Hayashi et al., Chemistry Letters 1984, 1747-1750. Glycosyl fluorides carrying complex oligosaccharide moieties may be produced via biotechnological methods such as that described in WO 2021170620 (A1). A method for the production of sphingolipid acceptors such as the sphingolipid of formula (8) is, for example, described in WO 2022158993 (A1).

A method for the synthesis of glycosides such as 4,6-disubstituted 1,3,5-triazyn-2-yl-glycosides such as glycosides of formula (5), or (9) is, for example, described in Tanaka et al., J. Appl. Glycosci. 2009, 56, 83-88. Alternatively, 4,6-disubstituted 1,3,5-triazyn-2-yl-glycosides such as glycosides of formula (5), or (9) may also be synthesized according to the procedure disclosed in the present invention.

The glycosphingolipid to be produced may be represented by a glycosphingolipid of formula (24), or a salt thereof:

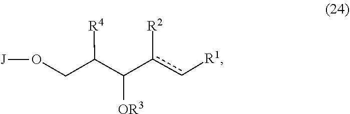

wherein
J is as defined as for the glycosyl donor of formula (2), and
R¹, R², R³, R⁴, and the bond [symbol] are as defined as for the compound of formula (4).

In some embodiments, the glycosphingolipid to be produced is represented by a glycosphingolipid of formula (25), or a salt thereof:

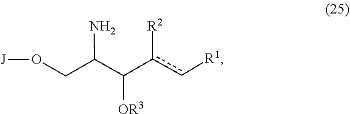

wherein
J is as defined as for the glycosyl donor of formula (2), and
R¹, R³, R⁴, and the bond [symbol] are as defined as for the compound of formula (4).

In some embodiments, J of the glycosyl donor of formula (2) and of the glycosphingolipid formula (24) or (25) is the glycosyl moiety of a human milk oligosaccharide, wherein the human milk oligosaccharide is preferably selected from the group consisting of lacto-N-tetraose (LNT), lacto-N-Neotetraose (LNnT), lacto-N-hexaose (LNH), lacto-N-neohexaose (LNnH), 2′-fucosyllactose (2′FL), 3-fucosyllactose (3-FL), difucosyllactose (DFL), lacto-N-fucopentaose I (LNFP-I), lacto-N-fucopentaose II (LNFP-II), lacto-N-fucopentaose III (LNFP-III), lacto-N-fucopentaose V (LNFP-V), lacto-N-difucohexaose I (LNDFH-I), 3′sialyllactose (3′SL), 6′sialyllactose (6′SL), 3-fucosyl-3′-sialyllactose (FSL), sialyllacto-N-tetraose a (LSTa), sialyllacto-N-tetraose b (LSTb), sialyllacto-N-tetraose c (LSTc), and disialyllacto-N-tetraose (DSLNT).

In the context of the present invention the glycosyl moieties of LNT, LNnT, LNH, LNnH, 2′FL, 3FL, DFL, LNFP-I, LNFP-II, LNFP-III, LNFP-V, LNDFH-I, 3′SL, 6′SL, FSL, LSTa, LSTb, LSTc, and DSLNT may be represented by the following formulas:

Galβ1-3GlcNAcβ1-3Galβ1-4Glc1-,
Galβ1-4GlcNAcβ1-3Galβ1-4Glc1-,
Galβ1-3GlcNAcβ1-3(Galβ1-4GlcNAcβ1-6)Galβ1-4Glc1-,
Galβ1-4GlcNAcβ1-3Galβ1-4GlucNAcβ1-3Galβ1-4Glc1-,
Fucα1-2Galβ1-4Glc1-,
Galβ1-4(Fucα1-3)Glc1-,
Fucα1-2Galβ1-4(Fucα1-3)Glc1-,
Fucα1-2Galβ1-3GlcNAcβ1-3Galβ1-4Glc1-,
Galβ1-3(Fucα1-4)GlcNAcβ1-3Galβ1-4Glc1-,
Galβ1-3(Fucα1-3)GlcNAcβ1-3Galβ1-4Glc1-,
Galβ1-3GlcNAcβ1-3Galβ1-4(Fucα1-3)Glc1-,
Fucα1-2Galβ1-3(Fucα1-4)GlcNAcβ1-3Galβ1-4Glc1-,
Neu5Acα2-3Galβ1-4Glc1-,
Neu5Acα2-6Galβ1-4Glc1-,
Neu5Acα2-3Galβ1-4(Fucα1,3)Glc1-,
Neu5Acα2-3Galβ1-3GlcNAcβ1-3Galβ1-4Glc1-,
Galβ1-3(Neu5Acα2-6)GlcNAcβ1-3Galβ1-4Glc1-,
Neu5Acα2-6Galβ1-3GlcNAcβ1-3Galβ1-4Glc1-,
Neu5Acα2-3Galβ1-3(Neu5Acα2-6)GlcNAcβ1-3Galβ1-4Glc1-,

respectively.

In some embodiments, A of the glycosyl donor of formula (2) and of the glycosphingolipid formula (24) or (25) is the oligosaccharide portion of a ganglioside selected from GM1a, GM1b, GD1a, GD1b, GD3, GT1b, GT3, GQ1b, GM3, GM4.

In the context of the present invention the oligosaccharide portion of GM1a, GM1b, GD1a, GD1b, GD3, GT1b, GT3, GQ1b, and GM4 may be represented by the following formulas:

Galβ1-3GalNAcβ1-4(Neu5Acα2-3)Galβ1-4Glc1-,
Neu5Acα2-3Galβ1-3GalNAcβ1-4Galβ1-4Glc1-,
Neu5Acα2-3Galβ1-3GalNAcβ1-4(Neu5Acα2-3)Galβ1-4Glc1-,
Galβ1-3GalNAcβ1-4(Neu5Acα2-8Neu5Acα2-3)Galβ1-4Glc1-,
Neu5Acα2-8Neu5Acα2-3Galβ1-4Glc1-,
Neu5Acα2-3Galβ1-3GalNAcβ1-4(Neu5Acα2-8Neu5Acα2-3)Galβ1-
4Glc1-,
Neu5Acα2-8Neu5Acα2-8Neu5Acα2-3Galβ1-4Glc1-,
Neu5Acα2-8Neu5Acα2-3Galβ1-3GalNAcβ1-4(Neu5Acα2-
8Neu5Acα2-3)Galβ1-4Glc1,
Neu5Acα2-3Gal1-,

respectively.

The glycosphingolipid to be produced may in some embodiments be lactosyl sphingosine, lactosyl dihydrosphingosine, lactosyl phytosphingosine, lactosyl ceramide or 3′SL sphingosine.

The glycosphingolipid to be produced may in some embodiments be a ganglioside. The ganglioside is preferably a human brain ganglioside, especially GD3, GM1, GD1a, GD1b, GT1b, GM3, GQ1b or GM4.

In some embodiments, the inventive method further comprises a glycosylation step of the glycosyl moiety. For example, if the inventive polypeptide catalyzes the synthesis of lactosyl sphingosine, the lactosyl sphingosine may be further reacted in the presence of a glycosyltransferase, such as a sialyltransferase.

The present invention further discloses an isolated nucleic acid comprising a nucleic acid sequence encoding an inventive polypeptide.

The isolated nucleic acid is in some embodiments a DNA sequence of SEQ ID NO: 20. The isolated nucleic acid is in some embodiments a nucleic acid sequence which is at least 70% identical with SEQ ID NO: 20, preferably a nucleic acid sequence which is at least 80% identical with SEQ ID NO: 20, more preferably a nucleic acid sequence which is at least 90% identical with SEQ ID NO: 20, even more preferably a nucleic acid sequence which is at least 95% identical with SEQ ID NO: 20.

As used herein, the term “isolated” means that the nucleic acid or polypeptide has been essentially removed from other biological materials with which it is naturally associated, or essentially free from other biological materials derived, e.g., from a recombinant host cell that has been genetically engineered to express the polypeptide of the invention. The term “isolated” also includes nucleic acids or polypeptides that have been chemically synthesized and are thus substantially uncontaminated by other proteins or nucleic acids.

The term “nucleic acid sequence” in the context of the invention refers to a DNA fragment, which is either double-stranded or single stranded, or to a product of transcription of said DNA fragment, and/or to an RNA fragment. A nucleic acid sequence may be naturally present in a cell where it is expressed naturally as a part of cell genomic sequence (termed as “endogenous nucleic acid sequence”) or may be introduced into a cell by recombinant nucleic acid techniques (termed as “heterologous nucleic acid sequence”). A heterologous nucleic acid sequence may be a nucleic acid sequence that originates from a source foreign to the particular host cell, or it may originate from the same source. If derived from the same source, the nucleic sequence may be modified from its original form. Thus, a heterologous nucleic acid sequence in a cell also includes nucleic acid sequences that are endogenous to the particular cell. These may be fragments of the original genomic sequence that were or were not subjected to one or more modifications. Non-limiting examples endogenous nucleic acid sequences that are subjected one or modifications include constructs comprising an endogenous nucleic acid sequence that operably linked to a promoter and/or another regulatory sequence that is not naturally linked to said sequences in the genome, or the nucleic acid sequences that comprise nucleobase substitutions introduced by site-directed mutagenesis. A heterologous nucleic sequence of the invention also includes recombinant DNA sequence.

A heterologous nucleic acid sequence can be expressed in the cell transiently, e.g. as plasmid borne, or stably, e.g. from a genome integrated expression cassette. One expression vector can be used for one or several expression cassettes or more than one expression vector can be used for more than one expression cassette. Heterologous nucleic acid sequences according to the present invention can also be inserted into the chromosome of the cell, using methods known to those skilled in the art, including homologous recombination, site-specific recombination or transposon-mediated gene transposition. The CRISPR technology may also be used to insert one or more heterologous nucleic acid sequences or one or more expression cassettes into a specific locus of the chromosome of the cell. Combinations of expression cassettes in extrachromosomal vectors and expression cassettes inserted into a host cell chromosome can also be used.

Commonly known recombinant nucleic acid techniques are described e.g. in Sambrook et al., Molecular Cloning: A Laboratory Manual, 2^{nd} Ed., Cold Spring Harbor Laboratory Press, Cold Spring Harbor, N.Y. (1989).

The term “wild type” includes entities having a structure and/or activity as found in a normal (as contrasted with e.g. mutant or altered) state or context. Wild type genes and polypeptides often exist in multiple different forms (e.g., alleles).

In some embodiments, the method according to the present invention is performed in vivo in a genetically modified cell. The genetically modified cell according to the present invention may be prokaryotic or eukaryotic. The host cell used in the method according to the present invention is typically a microorganism, such as a bacterium or a yeast. One preferred bacterium species is Escherichia coli (E. coli). One preferred yeast species is Wickerhamomyces ciferrii (W. ciferrii).

The expression “genetically modified cell” means that at least one alteration in the DNA sequence has been performed in the genome of the cell in order to give that cell a desired specific phenotype. The alteration in the DNA may e.g. be an introduction or a deletion of a DNA fragment in the genome, or an introduction of an expression vector carrying an endogenous or foreign gene in the cell. The alteration in the DNA sequence is herein especially achieved by the expression of a heterologous nucleic acid sequence, in particular a heterologous nucleic acid sequence encoding an endoglycoceramide synthase enzyme. Genome editing may be performed e.g. by commonly known recombinant nucleic acid techniques as e.g. described in Sambrook et al., Molecular Cloning: A Laboratory Manual, 2^{nd} Ed., Cold Spring Harbor Laboratory Press, Cold Spring Harbor, N.Y. (1989). The CRISPR technology may also be used to perform genetic modifications.

The nucleic acid sequences according to the present invention comprise or consists of a coding DNA sequence, i.e. a gene, a derivative of a gene or a transcription product of a gene, or a synthetic construct substantially identical to a gene. A derivative of a gene includes a nucleic acid sequence that is a fragment of a gene or a nucleic acid sequence that contains one or more mutations and/or deletions as compared to the original gene, or a cDNA; the mutations or deletions must not strongly impair the function of the encoded enzyme. A derivative of a gene is preferably at least 60% identical to a gene, more preferably at least 90% identical to a gene, even more preferably at least 95% identical to a wildtype gene. The value for gene identity is typically generated when two or more nucleotide sequences are compared and aligned for maximum correspondence, as measured using one of algorithms accepted for this purpose in the art, e.g. as the following sequence comparison algorithms. A synthetic construct substantially identical to a gene may be produced by synthesis techniques known to the skilled person. Because of the degeneracy of the genetic code, a large number of functionally identical nucleic acids encode any given peptide or protein. For instance, the codons CGU, CGC, CGA, CGG, AGA and AGG all encode the amino acid arginine. Thus, at every position where an arginine is specified by a codon, the codon can be altered to any of the corresponding codons described without altering the encoded peptide or protein. A derivative of a gene or a synthetic construct substantially identical to a gene is a nucleic acid sequence is in one embodiment codon-optimized for expression in the genetically modified cell according to the present invention. “Codon optimization” takes advantage of the degeneracy of codons, as exhibited by the multiplicity of three-base pair codon combinations that specify an amino acid, and generally includes a process of modifying a nucleic acid sequence for enhanced expression in particular host cells by replacing at least one codon of the native sequence with a codon that is more frequently or most frequently used in the genes of the host cell while maintaining the native amino acid sequence. For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)). Examples of algorithms that are suitable for determining percent sequence identity and sequence similarity are the BLAST and BLAST 2.0 algorithms, which are described in Altschul el al. (1990) J. Mol. Biol. 215:403-410 and Altschuel et al. (1977) Nucleic Acids Res. 25:3389-3402, respectively. Software for performing BLAST analyses is publicly available through the National Center for Biotechnology Information (www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands.

“Complementarity” of nucleic acids means that a nucleotide sequence in one strand of nucleic acid, due to orientation of its nucleobase groups, forms hydrogen bonds with another sequence on an opposing nucleic acid strand. The complementary bases in DNA are typically A with T and C with G. In RNA, they are typically C with G and U with A. Complementarity can be perfect or substantial/sufficient. Perfect complementarity between two nucleic acids means that the two nucleic acids can form a duplex in which every base in the duplex is bonded to a complementary base by Watson-Crick pairing. “Substantial” or “sufficient” complementary means that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations to predict the Tm (melting temperature) of hybridized strands, or by empirical determination of Tm by using routine methods.

In some embodiments, the glycosyl donor, the sphingolipid acceptor, the glycosphingolipid, as well as the glycoside and the saccharide, as described above, may be produced or utilized in the form of salts, preferably in the form of pharmaceutical acceptable salts.

In some embodiments, the salts comprising the following cations: Na+, K+, Mg2+, Ca2+, NH4+, Li+.

In some embodiments, the salts comprising the following anions: Cl−, Br−, CH3CO²−, CO3²−, SO4²−, HPO⁴−.

### EXAMPLES

### General Methods and Material

¹H NMR and ¹³C NMR was recorded with a Bruker WM-300S (300/75.1 MHz) spectrometer.

¹H and ¹³C chemical shifts are given in ppm (δ) relative to H₂O (δ=4.79), or MeOH (δ=3.34) as internal standard.

LCMS analysis was performed with a Shimadzu ECO 2020 LC system coupled with a Shimadzu LCMS-2020 system.

TLC-analysis was performed with silica gel TLC-plates (Merck, Silica gel, F254) with detection by carring (±140° C.) with ammonium molybdate (25 g/L) and cerium ammonium sulfate (10 g/L) in 10% H₂SO₄.

Column chromatography was performed on Silica gel 60 (220-440 mesh ASTM, Fluka), or on Sfär Bio C18-D column.

EGC synthases were expressed from E. coli strains following methods described in Caines et al., J. Biol. Chem. 2007, 282, 14300-14308, or in Vaughan et al., J. Am. Chem. Soc. 2006, 128, 6300-6301, or in Han et al., J. Biol. Chem. 2017, 292, 4789-4800. 5-20 mg/mL of a cell-free extract powder form comprising the respective enzyme were added to the respective reactions.

### Example 1: Strain Design and Enzyme Expression and Purification

A synthetic DNA sequence encoding the EGC synthase was cloned into a plasmid according to standard molecular cloning techniques and transformed into an E. coli host strain. Strain growth and enzyme purification were performed using standard expression and purification methods.

### Example 2: Km Determination

The assay mixture contained 1 M sodium acetate buffer at pH 5.5, 20 mg/mL sphingosine-base and EGCS enzyme. As a reference, EGC synthase of SEQ ID NO:17 was used. Six different concentrations of lactosyl fluoride (Lac-F) ranging from 5 mg/mL to 164 mg/mL were assayed. From every assay mixture, four time points were collected by quenching the reaction with 19× volume of DMSO. The samples were analyzed by HPLC-ELSD analysis and conversion rates were determined by linear regression.

TABLE 3
Km determination

| | | | | | | | Fold |
|---|---|---|---|---|---|---|---|
| | | | | Km Lac- | Keff (s⁻¹ | U/mg | improvement |
| Enzyme | Donor | Acceptor | Kcat (s⁻¹) | F (mM) ¹⁾ | M⁻¹) | CFE | to reference |
| SEQ ID NO: 4 | Lac-F | Sphingosine | 2.06 ± | 62 ± | 33.2 | 0.20 ± | 2.7 |
| | | | 0.01 | 16 | | 0.02 | |
| SEQ ID NO: 5 | Lac-F | Sphingosine | 2.28 ± | 254 ± | 9.0 | 0.61 ± | 8.2 |
| | | | 0.04 | 50 | | 0.05 | |
| SEQ ID NO: 8 | Lac-F | Sphingosine | 2.8 ± | 39.3 ± | 71.2 | 0.60 ± | 8.1 |
| | | | 0.3 | 6.9 | | 0.03 | |
| Reference | Lac-F | Sphingosine | 0.76 ± | 69 ± | 11.0 | 0.074 ± | 1.0 |
| (SEQ ID NO: | | | 0.01 | 5 | | 0.002 | |
| 17)) | | | | | | | |

### Example 3: In-Vitro Enzymatic Glycosylation of Sphingolipids with Glycosyl Fluoride Donors

### Example 3.1: Production of Lac-Ceramide, Lac-Phytosphingosine, Lac-Dihydrosphingosine and 3′SL-Sphingosine

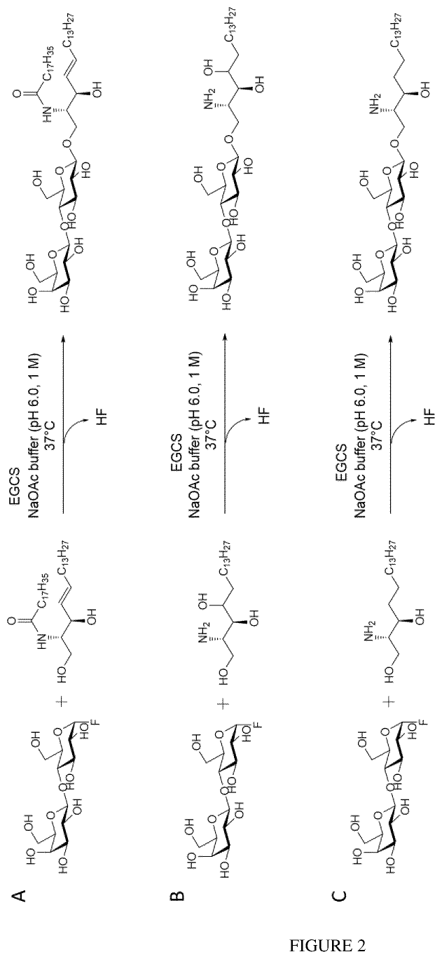

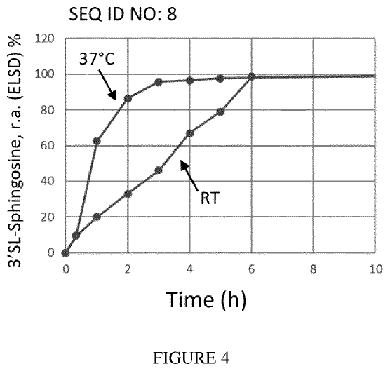

Reaction contained: 200-600 mM of glycosyl donor, EGCS (SEQ ID NO: 3), 1 M NaOAc buffer at pH 6.0, 100-600 mM acceptor. Reaction was executed at 37° C. or, as a comparison, at 22° C., until desired conversion was reached. Product analysis was done by ELSD (% rel. conversion) and LC-MS using standard conditions. Reaction schemes of Lac-ceramide, Lac-phytosphingosine and Lac-dihydrosphingosine are shown in FIG. 2. Production of 3′SL-sphingosine is shown in FIG. 4.

### Example 3.2: Production of LNnT-Sphingosine

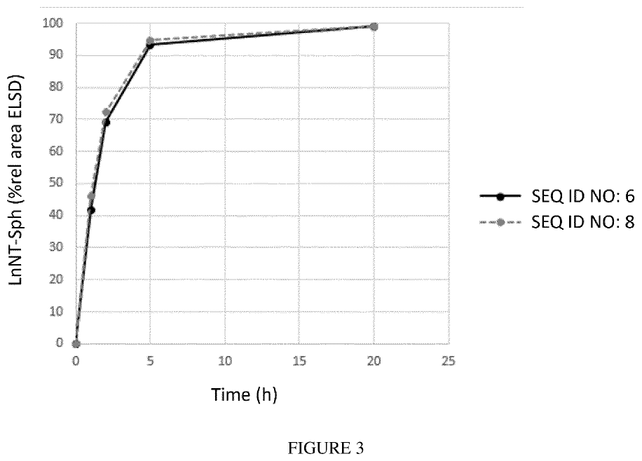

Reaction conditions: 200 mg/mL donor (˜300 mM LNNT-F); EGCS enzyme 1 M NaOAc buffer; 60 mg/mL Sph (˜200 mM H2SO4-Sph); reaction was executed at pH 6.0 (stabilized with 2 M NaOH), 1500 rpm stirring and 37° C. Product analysis was done by measuring conversion of Sph by ELSD, as shown in FIG. 3)

LC-MS: ESI-MS calculated for [C₄₄H₈₀N₂O₂₂]: 988, found: 989 [M+H]⁺, 987 [M−H]⁻.

### Example 3.3: Production of Lactosyl-6-hydroxysphingosine

Reaction contained: 30 mg/mL 6-OH-Sph, 300 mg/mL Lac-F, enzyme, 1 M NaOAc.

Reactions were executed at pH 6.0, 37° C. and 1500 rpm stirring. Product analysis was done by ELSD (% rel. conversion) and LC-MS using standard conditions for Lac-Sph analysis.

TABLE 4
Comparison of conversion rates of different enzymes (“catalyst”) for
the production of lactosyl-6-hydroxysphingosine

| | Time | Conversion |
|---|---|---|
| Catalyst | [h] | [% ELSD area product] |
| SEQ ID NO: 15 | 1 | 3.7 |
| SEQ ID NO: 17 | 1 | 99.4 |
| SEQ ID NO: 6 | 1 | 100 |
| SEQ ID NO: 8 | 1 | 100 |
| RhoT Synthase | 1 | 4.2 |
| NocP Synthase | 1 | 0 |
| SEQ ID NO: 18 | 1 | 99.3 |
| — | 1 | 0 |
| SEQ ID NO: 15 | 2 | 9.1 |
| SEQ ID NO: 17 | 2 | 100 |
| SEQ ID NO: 6 | 2 | 100 |
| SEQ ID NO: 8 | 2 | 100 |
| RhoT Synthase | 2 | 8.8 |
| NocP Synthase | 2 | 0 |
| SEQ ID NO: 18 | 2 | 99.8 |
| — | 2 | 0 |

RhoT Synthase: mutated (E342S) enzyme from Rhodococcus triatomea endoglycoceramidase, wildtype enzyme having the accession number M2W5L3_9NOCA.
NocP Synthase: mutated (E341S) enzyme from Nocardia puris endoglycoceramidase, wildtype enzyme having the accession number A0A366DJP0.

LC-MS: ESI-MS calculated for [C₃₀H₅₇NO₁₃]: 639, found: 640 [M+H]⁺, 638 [M−H]⁻.

### Example 3.4: Production of LNT-sphingosine

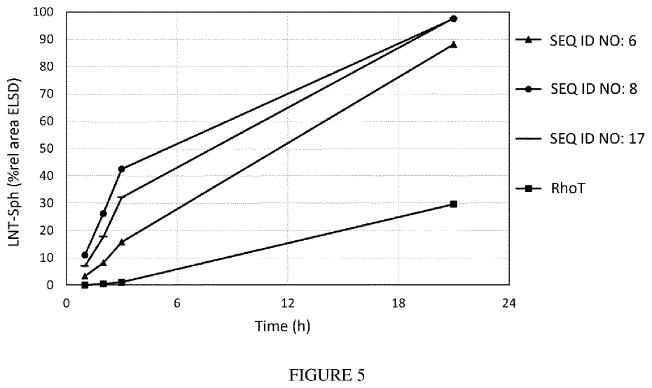

Reaction conditions: 300 mM LNT-F, 1 M NaOAc buffer, 200 mM H₂SO₄-Sph, enzyme. Reaction was executed at pH 6.0 (stabilized with 5 M NaOH), 1000 rpm stirring and 37° C. Product analysis was done by measuring conversion of Sph by ELSD, as shown in FIG. 5)

LC-MS: ESI-MS calculated for [C₄₄H₇₉N₂O₂₂]: 988.52 found: 989.50 [M+H]⁺, 987.50 [M−H]⁻.

### Example 4: Synthesis of 4,6-dimethoxy 1,3,5-triazin-2-yl-glycosides

### Example 4.1: General Procedure for the Synthesis of Triazinyl Glycosides

A saccharide (1 eq.), 2-chloro-4,6-dimethoxy-1,3,5-triazine (1.7 eq.), NaHCO₃ (1.65 eq.), and 4-methylmorpholine (0.15 eq.) were suspended in water (5-50 mL). The suspension was stirred at a temperature between 20° C. to 25° C. until a TLC-analysis showed complete consumption of the starting material. Subsequently, ethanol or acetonitrile was added to the reaction mixture. The resulting suspension was filtered, washed with ethanol or acetonitrile, and dried in vacuo to obtain the final product.

### 4.2 Synthesis of 4,6-dimethoxy-1,3,5-triazin-2-yl β-D-galactopyranosyl-(1→4)-β-D-glucopyranoside (14)

Compound (14) was obtained from lactose following the general procedure described in example 4.1.

¹H NMR (D₂O) δ 5.94 (d, J=8.0 Hz, 1H), 4.49 (d, J=7.8 Hz, 1H), 4.04 (s, 6H), 4.02-3.92 (m, 2H), 3.90-3.65 (m, 9H), 3.58 (dd, J=10.0, 7.8 Hz, 1H)):

¹³C NMR ((D₂O) δ 173.33, 171.93, 102.93, 96.84, 77.67, 75.56, 75.37, 74.07, 72.52, 71.80, 70.95, 68.56, 61.05, 59.70, 55.91):

ESI-MS: calculated for [C₁₇H₂₇N₃O₁₃]: 481, found: 482 [M+H]⁺, 504 [M+Na]⁺.

### 4.3 Synthesis of dimethoxy-1,3,5-triazin-2-yl β-D-galactopyranosyl-(1→4)-O-2-(acetylamino)-2-deoxy-β-D-glucopyranosyl-(1→3)-O-β-D-galactopyranosyl-(1→4)-β-D-glucopyranoside (15)

Compound (15) was obtained from lacto-N-neotetraose following the general production procedure described in example 4.1.

¹H NMR (D₂O) δ: 5.95 (d, J=8.0 Hz, 1H), 4.74 (d, J=8.3 Hz, 1H), 4.51 (d, J=4.8 Hz, 1H), 4.49 (d, J=4.7 Hz, 1H), 4.19 (d, J=3.5 Hz, 1H), 4.05 (s, 6H), 4.04-3.93 (m, 5H), 3.91-3.52 m, 19H), 2.07 (s, 3H).

¹³C NMR (D₂O) δ: 174.87, 173.29, 171.89, 102.90, 102.84, 102.74, 96.79, 81.98, 78.14, 77.57, 75.52, 75.32, 74.87, 74.53, 74.02, 72.48, 72.15, 71.74, 70.94, 69.93, 68.52, 68.32, 61.00, 60.95, 59.84, 59.64, 55.87, 55.16, 22.15).

ESI-MS: calculated for [C₃₁H₅₀N₄O₂₃]: 846, found: 847 [M+H]⁺, 869 [M+Na]⁺.

### 4.4. Synthesis of 4,6-dimethoxy-1,3,5-triazin-2-yl β-D-galactopyranosyl-(1→3)-O-2-(acetylamino)-2-deoxy-β-D-glucopyranosyl-(1→3)-O-β-D-galactopyranosyl-(1→4)-β-D-glucopyranoside (16)

Compound (16) was obtained from lacto-N-tetraose following the general production procedure described in example 4.1.

¹H NMR (D₂O) δ: 5.89 (d, J=8.1 Hz, 1H), 4.69 (d, J=8.4 Hz, 1H), 4.42 (d, J=7.8 Hz, 1H), 4.40 (d, J=7.7 Hz, 1H), 4.12 (d, J=3.3 Hz, 1H), 3.99 (s, 6H), 3.97-3.40 (m, 24H), 1.99 (s, 3H).

¹³C NMR (D₂O) δ: 174.91, 173.26, 171.84, 103.43, 102.84, 102.54, 96.73, 81.91, 77.47, 75.47, 75.21, 75.10, 74.84, 73.96, 72.38, 71.68, 70.60, 69.92, 68.45, 68.36, 68.26, 63.89, 60.97, 60.40, 59.57, 55.81, 54.62, 22.14.

ESI-MS: calculated for [C₃₁H₅₀N₄O₂₃]: 846, found: 847 [M+H]⁺, 869 [M+Na]⁺.

### 4.5. Synthesis of 4,6-dimethoxy-1,3,5-triazin-2-yl α-L-fucopyranosyl-(1→2)-O-β-D-galactopyranosyl-(1→4)-β-D-glucopyranoside (17)

Compound (17) was obtained from 2′-fucosyllactose following the general production procedure described in example 4.1.

¹H NMR (D₂O) δ: 5.89 (d, J=7.8 Hz, 1H), 5.34 (d, J=3.3 Hz, 1H), 4.58 (d, J=7.5 Hz, 1H), 4.26 (m, 1H), 4.05 (s, 6H), 4.02-3.68 (m, 15H), 1.27 (d, J=6.8 Hz, 3H).

¹³C NMR (D₂O) δ: 173.34, 171.89, 100.27, 99.40, 96.90, 76.40, 75.98, 75.26, 73.97, 73.59, 71.89, 71.70, 69.63, 69.13, 68.22, 66.98, 61.12, 59.79, 55.93, 15.31.

ESI-MS: calculated for [C₂₃H₃₇N₃O₁₇]: 627, found: 628 [M+H]⁺, 650 [M+Na]⁺.

### 4.6. Synthesis of 4,6-dimethoxy-1,3,5-triazin-2-yl α-L-fucopyranosyl-(1→3)-O-[β-D-galactopyranosyl-(1→4)]-β-D-glucopyranoside (18)

Compound (18) was obtained from 3-fucosyllactose following the general production procedure described in example 4.1.

ESI-MS: calculated for [C₂₃H₃₇N₃O₁₇]: 627, found: 628 [M+H]⁺, 650 [M+Na]⁺.

### 4.7. Synthesis of 4,6-dimethoxy-1,3,5-triazin-2-yl α-N-acetylneuraminosyl-(2→3)-O-β-D-galactopyranosyl-(1→4)-β-D-glucopyranoside (19)

Compound (19) was obtained from 3′-sialyllactose following the general production procedure described in example 4.1.

¹H NMR ((D₂O) δ 5.90 (d, J=8.1 Hz, 1H), 4.53 (d, J=8.0 Hz, 1H), 4.09 (dd, J=9.9, 3.1 Hz, 2H), 3.99 (s, 6H), 3.98-3.49 (m, 18H), 2.73 (dd, J=12.4, 4.7 Hz, 1H), 2.00 (s, 3H), 1.77 (t, J=12.1 Hz, 1H)):

¹³C NMR ((D₂O) δ 174.98, 173.86, 173.31, 171.91, 102.61, 99.77, 96.81, 77.46, 75.54, 75.45, 75.17, 74.00, 72.85, 71.76, 69.34, 68.33, 68.07, 67.44, 62.55, 61.02, 59.62, 55.86, 51.65, 39.62, 22.00):

ESI-MS: calculated for [C₂₈H₄₄N₄O₂₁]: 772, found: 773 [M+H]⁺, 795 [M+Na]⁺.

### 4.8 Synthesis of 4,6-dimethoxy-1,3,5-triazin-2-yl α-N-acetylneuraminosyl-(2→6)-O-β-D-galactopyranosyl-(1→4)-β-D-glucopyranoside (20)

Compound (20) was obtained from 6′-sialyllactose following the general production procedure described in example 4.1.

¹H NMR (D₂O) δ: 5.96 (d, J=8.2 Hz, 1H), 4.50 (d, J=7.8 Hz, 1H), 4.21-4.12 (m, 1H), 4.06 (s, 6H), 4.01-3.54 (m, 19H), 2.77 (dd, J=12.4, 4.7 Hz, 1H), 2.07 (s, 3H), 1.79 (t, J=12.2 Hz, 1H).

¹³C NMR (D₂O) δ 174.88, 173.46, 173.31, 171.94, 103.21, 100.27, 96.71, 78.85, 75.39, 74.31, 73.70, 72.51, 72.32, 71.80, 71.71, 70.75, 68.50, 68.36, 63.58, 62.62, 59.82, 55.88, 51.77, 40.10, 22.04.

ESI-MS: calculated for [C₂₈H₄₄N₄O₂₁]: 772, found: 773 [M+H]⁺, 795 [M+Na]⁺.

### 4.9. Synthesis of 4,6-dimethoxy-1,3,5-triazin-2-yl β-D-galactopyranosyl-(1→3)-O-2-(acetylamino)-2-deoxy-β-D-galactopyranosyl-(1→4)-O-[α-N-acetylneuraminosyl-(2→3)]-O-β-D-galactopyranosyl-(1→4)-β-D-glucopyranoside (21)

Compound (21) was synthesized from β-D-galactopyranosyl-(1→3)-O-2-(acetylamino)-2-deoxy-β-D-galactopyranosyl-(1→4)-O-[α-N-acetylneuraminosyl-(2→3)]-O-β-D-galactopyranosyl-(1→4)-α/β-D-glucopyranose following the general production procedure described in example 4.1.

ESI-MS: calculated for [C₄₂H₆₇N₅O₃₁]: 1137, found: 1138 [M+H]⁺, 1160 [M+Na]⁺.

### 4.10. Synthesis of 4,6-dimethoxy-1,3,5-triazin-2-yl O—(N-acetyl-α-neuraminosyl)-(2→8)-O—(N-acetyl-α-neuraminosyl)-(2→3)-O-β-D-galactopyranosyl-(1→4)-β-D-glucopyranoside (22)

Compound (22) was synthesized from α-N-acetylneuraminosyl-(2→8)-O-α-N-acetylneuraminosyl-(2→3)-O-β-D-galactopyranosyl-(1→4)-α/β-D-glucopyranose following the general procedure described in example 4.1.

¹H NMR (D₂O) δ: 5.89 (d, J=7.8 Hz, 1H), 4.51 (d, J=7.5 Hz, 1H), 4.15-4.02 (m, 1H), 3.98 (s, 6H), 3.95-3.47 (m, 26H), 2.78-2.56 (m, 2H), 2.02 (s, 3H), 1.98 (s, 3H), 1.69 (t, J=12.0 Hz, 2H).

ESI-MS: calculated for [C₃₉H₆₁N₅O₂₉]: 1063, found: 1064 [M+H]⁺, 1086 [M+Na]⁺.

### 4.11. Synthesis 4,6-dimethoxy-1,3,5-triazin-2-yl α-L-fucopyranoside (23)

Compound 1q was synthesized from fucose following the general procedure described in example 4.1.

¹H NMR (D₂O) δ: 5.77 (d, J=7.3 Hz, 1H), 3.98 (s, 6H), 3.97-3.69 (m, 4H), 1.22 (d, J=6.4 Hz, 3H).

ESI-MS: calculated for [C₁₁H₁₇N₃O₇]: 303, found: 304 [M+H]⁺, 326 [M+Na]⁺.

### Example 5: In-Vitro Enzymatic Glycosylation of Sphingolipids with In-Situ Generation of the Glycosyl Fluoride Donor

### Example 5.1: General Procedure for Glycosylation of with In-Situ Generation of the Glycosyl Fluoride Donor

Enzymatic glycosylation reactions were performed in 1 M NaOAc buffer (pH set to 6.0) containing 2 M of KF. A typical reaction mixture contained 200 mM of a 4,6-dimethoxy-1,3,5-triazin-2-yl glycoside, 50 mM of a sphingolipid acceptor, and enzyme of SEQ ID NO: 4 to 6, 8, 15 or 17 in a total reaction volume of 1 mL. Typically, the reaction proceeds to 25-85% conversion within 67 to 366 hours at 37° C. and continuous stirring. Glycosylation of the sphingolipid was monitored by LCMS analysis (for methods and conditions see Example 6).

### 5.2 Production of β-D-galactopyranosyl-(1→4)-β-D-glucopyranosyl-(1→1′)-D-erythro-sphingosine (lactosylsphingosine)

Lactosylsphingosine was produced from glycoside (14) and D-erythro-sphingosine following the general procedure described in Example 5.1.

LC Analysis: Rt=2.22 min, identical to reference standard.

### 5.3 Production of α-1-fucopyranosyl-(1→2)-O-β-D-galactopyranosyl-(1→4)-β-D-glucopyranosyl(1→1′)-D-erythro-sphingosine (2′-fucosyllactosyl-sphingosine)

2′-Fucosyllactosyl-D-erythro-sphingosine was produced from glycoside (17) and d-erythro-sphingosine, following the general procedure described in Example 5.1.

LC/MS: Rt=3.20 min; ESI-MS calculated for [C₃₆H₆₇NO₁₆]: 769, found: 770 [M+H]⁺, 768 [M−H]⁻.

### 5.4. Production of β-D-galactopyranosyl-(1→3)-O-2-(acetylamino)-2-deoxy-β-D-glucopyranosyl-(1→3)-O-β-D-galactopyranosyl-(1→4)-β-D-glucopyranosyl-(1→1′)-D-erythro-sphingosine (Lacto-N-tetraosyl-D-erythro-sphingosine)

Compound Lacto-N-neotetraosyl-D-erythro-sphingosine was produced from glycoside (16) and D-erythro-sphingosine, following the general procedure described in Example 5.1.

LC/MS: Rt=7.16 min; ESI-MS calculated for [C₄₄H₈₀N₂O₂₂]: 988, found: 989 [M+H]⁺, 987 [M−H]⁻.

### 5.5. Production of β-D-galactopyranosyl-(1→4)-O-2-(acetylamino)-2-deoxy-β-D-glucopyranosyl-(1→3)-O-β-D-galactopyranosyl-(1→4)-β-D-glucopyranosyl-(1→1′)-D-erythro-sphingosine (Lacto-N-neotetraosyl-D-erythro-sphingosine)

Lacto-N-neotetraosyl-D-erythro-sphingosine was produced from glycoside (15) and D-erythro-sphingosine, following the general procedure described in Example 5.1.

LC/MS: Rt=3.00 min; ESI-MS calculated for [C₄₄H₈₀N₂O₂₂]: 988, found: 989 [M+H]⁺, 987 [M−H]⁻.

### 5.6. Production of α-N-acetylneuraminosyl-(2→6)-O-β-D-galactopyranosyl-(1→4)-β-D-glucopyranosyl-(1→1′)-D-erythro-sphingosine (6′-Sialyllactosyl-D-erythro-sphingosine)

6′-Sialyllactosyl-D-erythro-sphingosine was produced from glycoside (20) and D-erythro-sphingosine, following the general procedure described in Example 5.1.

After completion, the enzyme was denatured by adding MeOH (400 mL) to the reaction mixture. The precipitate was filtered off, and the resulting solution was dried in vacuum to afford a solid residue. The solid residue was subjected to reverse phase chromatography using a RP-C18 column and a gradient of MeOH in H₂O to afford pure 6′-sialyllactosyl-D-erythro-sphingosine.

LC/MS: Rt=4.20 min; ESI-MS calculated for [C₄₁H₇₄N₂O₂₀]: 914, found: 915 [M+H]⁺, 913 [M−H]⁻.

¹H NMR (400 MHZ, CD₃OD) δ=5.87 (dt, J=15.4, 6.8 Hz, 1H), 5.49 (dd, J=15.4, 6.8 Hz, 1H), 4.45-4.25 (m, 3H), 4.10-3.19 (m, 22H), 2.93-2.68 (m, 1H), 2.18-2.06 (m, 2H), 1.66 (pt, J=12.0, 12.0 Hz, 1H), 1.52-1.11 (m, 24H), 0.90 (pt, J=6.8, 6.2 Hz, 3H) ppm.

¹³C NMR (101 MHZ, CD₃OD) δ=175.1, 136.7, 128.4, 104.9, 103.6, 101.6, 80.8, 76.6, 76.2, 75.8, 74.7, 74.3, 73.2, 70.9, 70.6, 70.1, 69.8, 67.1, 64.8, 61.9, 57.7, 56.7, 53.9, 49.6, 49.4, 49.2, 49.0, 48.8, 48.6, 48.4, 42.5, 33.4, 33.1, 30.8, 30.7, 30.6, 30.5, 30.4, 30.2, 23.7, 14.4 ppm.

### 5.7. Production of α-N-acetylneuraminosyl-(2→3)-O-β-D-galactopyranosyl-(1→4)-β-D-glucopyranosyl-(1→1′)-D-erythro-sphingosine (3′-Sialyllactosyl-D-erythro-sphingosine)

3′-Sialyllactosyl-D-erythro-sphingosine was produced from glycoside (19) and D-erythro-sphingosine, following the general procedure described in Example 5.1.

LC/MS: Rt=4.38 min; ESI-MS calculated for [C₄₁H₇₄N₂O₂₀]: 914, found: 915 [M+H]⁺, 913 [M−H]⁻.

### 5.8. Production of α-N-acetylneuraminosyl-(2→3)-O-β-D-galactopyranosyl-(1→4)-β-D-glucopyranosyl-(1→1′)-D-ribo-phytosphingosine (3′-Sialyllactosyl-D-ribo-phytosphingosine)

3′-Sialyllactosyl-D-ribo-phytosphingosine was produced from glycoside (19) and D-ribo-phytosphingosine, following the general procedure described in Example 5.1.

LC/MS: Rt=2.93 min; ESI-MS calculated for [C₄₁H₇₆N₂O₂₁]: 932, found: 933 [M+H]⁺, 931 [M−H]⁻.

### 5.9. Production of α-N-acetylneuraminosyl-(2→3)-O-β-D-galactopyranosyl-(1→4)-β-D-glucopyranosyl-(1→1′)-D-erythro-sphinganine (3′-Sialyllactosyl-D-erythro-sphinganine)

3′-Sialyllactosyl-D-erythro-sphinganine was produced from glycoside (19) and D-erythro-sphinganine, following the general procedure described in Example 5.1.

LC/MS: Rt=3.42 min; ESI-MS calculated for [C₄₁H₇₆N₂O₂₀]: 916, found: 917 [M+H]⁺, 915 [M−H]⁻.

### 5.10. Production of α-N-acetylneuraminosyl-(2→3)-O-β-D-galactopyranosyl-(1→4)-β-D-glucopyranosyl-(1→1′)-N-stearoyl-D-erythro-sphingosine (GM3)

GM3 was produced from glycoside (19) and N-stearoyl-D-erythro-sphingosine

(CER[N(18)S]) following the general procedure described in Example 5.1. The reaction was performed in the presence of triton-X (0.5 wt %) and cholate (0.1 mg/ml).

LCMS: Rt=12.38 min; ESI-MS calculated for [C₅₉H₁₀₈N₂O₂₁]: 1180, found: 1181 [M+H]⁺, 1179 [M−H]⁻.

### 5.11. Production of β-D-galactopyranosyl-(1→3)-O-2-(acetylamino)-2-deoxy-β-D-galactopyranosyl-(1→4)-O-[α-N-acetylneuraminosyl-(2→3)]-O-β-D-galactopyranosyl-(1→4)-β-D-glucopyranosyl-(1→1′)-D-erythro-sphingosine (GM1-D-erythro-sphingosine)

GM1-D-erythro-sphingosine was produced from glycoside (21) and D-erythro-sphingosine following the general procedure described in Example 5.1.

LC/MS: Rt=4.38 min; ESI-MS calculated for [C₅₅H₉₇N₃O₃₀]: 1279, found: 1280 [M+H]⁺, 1278 [M−H]⁻.

### 5.12. Production of —N-acetylneuraminosyl-(2→8)-O-α-N-acetylneuraminosyl-(2→3)-O-β-D-galactopyranosyl-(1→4)-β-D-glucopyranosyl-(1→1′)-D-erythro-sphingosine (GD3-D-erythro-sphingosine)

GD3-D-erythro-sphingosine was produced from glycoside (21) and D-erythro-sphingosine following the general procedure described in Example 5.1.

LC/MS: Rt=8.02 min; ESI-MS calculated for [C₅₂H₉₁N₃O₂₈]: 1205, found: 1206 [M+H]⁺, 1204 [M−H]⁻.

### Example 6: Analytic Methods for the In-Vitro Enzymatic Glycosylation of Sphingolipids with Glycosyl Fluoride Donors

LC/MS analysis was performed with a Shimadzu ECO 2020 LC system coupled with a Shimadzu LCMS-2020 system.

LC analysis was performed using a Merck Ascentis Express RP-Amide column (15 cm×4.6 mm, 2.7 μm). The eluent consisted of solvent D (2 mM ammonium formate, 2 mL formic acid, 75% v/v MeOH, 25% v/v ACN)-solvent C (2 mM formic acid in filtered ddH₂O), and the following gradient was applied as stated in the table below. The flow rate was 1 mL/min.

TABLE 5
LC-MS analysis for the in-vitro enzymatic glycosylation
of sphingolipids with glycosyl fluoride donors

| | | | MS: | MS: | | |
|---|---|---|---|---|---|---|
| | | Rt | Found | Found | MS: | Eluent |
| Donor | Acceptor | (min) | [M + H]⁺ | [M − H]⁻ | Calc. | Gradient |
| α-Lac- | sphingosine | 3.25 | 624.3 | 622.3 | 623.8 | 77% |
| F | | | | | | isocratic |
| | | | | | | (D in C) |
| α-Lac- | dihydrosphingosine | 2.49 | 626.3 | 624.3 | 625.4 | 77% |
| F | | | | | | isocratic |
| | | | | | | (D in C) |
| α- | phytosphingosine | 2.25 | 642.3 | 640.4 | 641.8 | 77% |
| Lac-F | | | [664.2]^{a} | [686.3]^{b} | | isocratic |
| | | | | | | (D in C) |

^{a}main mass ion detected was [M + Na]⁺;
^{b}main mass ion detected was [M + Formate]⁻

### Example 7. Analytic Methods for the In-Vitro Enzymatic Glycosylation of Sphingolipids with In-Situ Generation of the Glycosyl Fluoride Donor

Samples (25 μL) were taken from reaction mixtures, mixed with DMSO (950 μL) and subjected to centrifugation (16.000 rpm, 5 min) and analyzed with a Shimadzu ECO 2020 LC system coupled with a Shimadzu LCMS-2020 system. The LC analysis was performed using a Merck Ascentis Express RP-Amide column (15 cm×4.6 mm, 2.7 μm). The flow rate was 1 mL/min.

The following solvents where utilized: solvent A: 10 mM ammonium formate in H₂O;

solvent B: acetonitrile; solvent C: 2 mM formic acid in filtered ddH₂O; solvent D: 2 mM ammonium formate, 2 mL formic acid, 75% v/v MeOH, 25% v/v CAN.

TABLE 6
LC-MS analysis for the in-vitro enzymatic glycosylation of sphingolipids
with in-situ generation of the glycosyl fluoride donors

| Example | Solvent | Gradient |
|---|---|---|
| 5.1 | D in C | 77% isocratic |
| 5.2 | D in C | 70-95% |
| 5.3 | B in A | 30-70% |
| 5.4 | D in C | 70-95% |
| 5.5 | D in C | 70-95% |
| 5.6 | D in C | 70-95% |
| 5.7 | D in C | 77% isocratic |
| 5.8 | D in C | 77% isocratic |
| 5.9 | D in C | 80-98% |
| 5.10 | D in C | 70-95% |
| 5.11 | D in C | 50-95% |

### Example 8. General Procedure for the Enzymatic Production of Galactosyl-Sphingoid Bases with β-Galactosidase

Enzymatic reactions were performed in 1 M NaOAc buffer (pH set to 5.0). A typical reaction mixture contained 50-100 g/L of lactosyl sphingoid base (synthesised according to the general procedure of Example 3 or Example 5), 5 g/L of β-cyclodextrin, 5 g/L of β-galactosidase (e.g. from A. oryzae). Typically, the reaction proceeds up to 99% conversion within about 48 to about 72 hours at about 37° C. and continuous stirring. The enzymatic reaction was followed by LCMS analysis (for methods and conditions see Example 9).

### Example 9. Analytic Methods for the Enzymatic Production of Galactosyl-Sphingoid Bases with β-Galactosidase

Samples (25 μL) were taken from reaction mixtures, mixed with DMSO (950 μL) and subjected to centrifugation (16.000 rpm, 5 min) and analyzed with a Shimadzu ECO 2020 LC system coupled with a Shimadzu LCMS-2020 system. The HPLC analysis was performed using a Merck Ascentis Express RP-Amide column (15 cm×4.6 mm, 2.7 μm). For analysis of compounds the eluent consisted of solvent D (2 mM ammonium formate, 2 mL formic acid, 75% v/v MeOH, 25% v/v ACN)-solvent C (2 mM formic acid in filtered ddH₂O), and the following gradient was applied as stated in the table below. The flow rate was 1 mL/min. The MS analysis was performed under the following conditions: ESI positive ionization, vaporizer temperature 300° C.; LC-MS mode, 1:1 split of flow; ESI negative ionization, vaporizer temperature 300° C.; LC-MS mode, 1:1 split of flow.

TABLE 7
LC-MS analysis for the production of galactosyl-sphingoid bases with β-galactosidase.

| | | | | MS: | MS: | |
|---|---|---|---|---|---|---|
| | | | Rt | Found | Found | MS: |
| Substrate | Product | Gradient | (min) | [M + H]⁺ | [M − H]⁻ | Calc. |
| Lac-sphingosine | Glc-sphingosine | 77% | 2.33 | 462.4 | 460.4 | 461.3 |
| | | isocratic | | | | |
| | | (D in C) | | | | |
| Lac- | Glc- | 77% | 2.75 | 464.3 | 462.4 | 463.3 |
| dihydrosphingosine | dihydrosphingosine | isocratic | | | | |
| | | (D in C) | | | | |
| Lac- | Glc- | 77% | 2.4 | 480.3 | 478.3 | 479.3 |
| phytosphingosine | phytosphingosine | isocratic | | | | |
| | | (D in C) | | | | |
| Lac-ceramide | Glc-ceramide | 77% | 4.15 | 728.7 | 726.5 | 727.6 |
| | | isocratic | | | | |
| | | (D in C) | | | | |

1. A polypeptide
a. comprising an amino acid motif of formula (1): 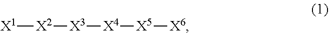
wherein
X¹ is an amino acid residue selected from for W;
X² is an amino acid residue selected from L, M, I, V or A;
X³ is an amino acid residue A, L or M;
X⁴ is an amino acid residue selected from G, A, S, N, Q, C, T, I, V, L or M;
X⁵ is an amino acid residue selected from F, T, M, L or S;
X⁶ is an amino acid residue selected from G, L or F;
and
b. having glycosynthase activity.

2. The polypeptide of claim 1, wherein the polypeptide has a catalytic activity to transfer a glycosyl moiety from a glycosyl donor to a sphingolipid acceptor.

3. The polypeptide of claim 1, wherein the polypeptide is a mutant endoglycoceramidase enzyme that has both a synthetic activity and a hydrolytic activity, wherein the synthetic activity of the enzyme prevails the hydrolytic activity.

4. The polypeptide of claim 1, wherein X³ is amino acid residue A.

5. The polypeptide of claim 1, wherein X⁴ is amino acid residue T or S.

6. The polypeptide of claim 1, having an amino acid sequence of any one of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or a variant and/or a fragment of any of them.

7. The polypeptide of claim 6, wherein the variant is an amino acid sequence that has at least 70% of sequence identity with the sequence of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12 or SEQ ID NO: 13.

8. The polypeptide of claim 1, having an amino acid sequence of any one of SEQ ID NO: 3 or SEQ ID NO: 8.

9. An isolated nucleic acid comprising a nucleic acid sequence encoding a polypeptide of claim 1.

10. The isolated nucleic acid of claim 9, comprising a nucleic acid sequence selected from the group consisting of:
a) a DNA sequence according to SEQ ID NO: 20, or a nucleic acid sequence complementary thereof;
b) a nucleic acid sequence comprising a sequence of a),
c) a nucleic acid sequence which is at least 70% identical with a sequence group a) or b), or a sequence complementary thereof, wherein said sequence comprises a nucleotide motif encoding the amino acid motif of formula (1).

11. The isolated nucleic acid of claim 9, wherein the sequence is recombinant nucleic acid sequence.

12. A genetically modified cell comprising a nucleic acid of claim 9.

13. The genetically modified cell of claim 12, capable of heterologous expression of a polypeptide according to claim 1.

14. Method of producing the polypeptide of claim 1, comprising
providing an isolated nucleic acid sequence of claim 10,
genetically modifying a cell to comprise the nucleic sequence of claim 10, and
expressing the polypeptide of claim 1 in the genetically modified cell.

15. (canceled)

16. A method of producing a glycosphingolipid, comprising:
reacting a glycosyl donor with a sphingolipid acceptor in the presence of a polypeptide according to claim 1, thereby
producing the glycosphingolipid,
wherein the glycosyl donor is either generated in situ or added to the reaction mixture.

17. (canceled)

18. The method according to claim 16, wherein the glycosyl donor is a compound of formula (2) 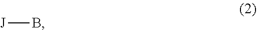
wherein
J is a glycosyl moiety,
B is selected from a fluoride, chloride, bromide, azide, formate, or iodide.

19-22. (canceled)

23. The method according to claim 16, wherein the sphingolipid acceptor is a compound of formula (3), or a salt thereof: 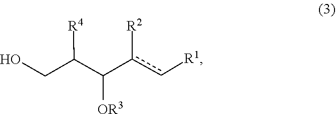
wherein
R¹ is H, aryl, or a C₁₋₂₀ alkyl, which may be saturated or contain one or more double and/or triple bonds, and/or which may contain one or more functional groups, the functional group being selected from the group consisting of a hydroxyl group, an alkoxy group, an acyloxy group, a primary, secondary, or tertiary amine, an acylamido group, a thiol, a thioether or a phosphorus-containing functional group;
R² is H or —OR⁵, wherein R⁵ is selected from hydrogen, a substituted or unsubstituted C₁₋₃ alkyl, or a substituted or unsubstituted C₂₋₄ acyl;
the bond [symbol] may be a double or a single bond when R² is H, or is a single bond when R² is —OR⁵;
R³ is H, a substituted or unsubstituted C₁₋₃ alkyl, or a substituted or unsubstituted C₁₋₃ acyl;
R⁴ is N₃ or NR⁶R⁷, wherein R⁶ and R⁷ are independently selected from H, a substituted or unsubstituted C₂₋₃₂ acyl, a substituted or unsubstituted aryl, a substituted or unsubstituted vinyl, or wherein R⁶ and R⁷ form a cyclic structure.

24-35. (canceled)
